SEQUENCE LISTING (38 sequences)

SEQ ID NO: 1 - protein, 127 aa, synthetic construct
MVSTPQFLVFLLFWIPASRGDILLTQSPAILSVSPGERVSFSCRASQNIGTSIHWYQQRTNGFPRLLIKYASESISGIPSRFSGSGSGTDFTLSINSVESEDIADYYCQQSYSWPFTFGSGTKLEIK

SEQ ID NO: 2 - protein, 11 aa, synthetic construct
RASQNIGTSIH

SEQ ID NO: 3 - protein, 7 aa, synthetic construct
YASESIS

SEQ ID NO: 4 - protein, 9 aa, synthetic construct
QQSYSWPFT

SEQ ID NO: 5 - protein, 23 aa, synthetic construct
DILLTQSPAILSVSPGERVSFSC

SEQ ID NO: 6 - protein, 15 aa, synthetic construct
WYQQRTNGFPRLLIK

SEQ ID NO: 7 - protein, 32 aa, synthetic construct
GIPSRFSGSGSGTDFTLSINSVESEDIADYYC

SEQ ID NO: 8 - protein, 10 aa, synthetic construct
FGSGTKLEIK

SEQ ID NO: 9 - protein, 134 aa, synthetic construct
MEWSGVFIFLLSVTADVHSQVQLQQSGAELVRPGTSVKISCKASGYTFTNYWLGWVKQRPGHGLEWIGNIFPGGGYTNYNEKFKGKAILTADTSSSTAYMQLSSLTSEDSGVYFCAREGVMDYWGQGTTLTVSS

SEQ ID NO: 10 - protein, 5 aa, synthetic construct
NYWLG

SEQ ID NO: 11 - protein, 17 aa, synthetic construct
NIFPGGGYTNYNEKFKG

SEQ ID NO: 12 - protein, 6 aa, synthetic construct
EGVMDY

SEQ ID NO: 13 - protein, 30 aa, synthetic construct
QVQLQQSGAELVRPGTSVKISCKASGYTFT

SEQ ID NO: 14 - protein, 14 aa, synthetic construct
WVKQRPGHGLEWIG

SEQ ID NO: 15 - protein, 32 aa, synthetic construct
KAILTADTSSSTAYMQLSSLTSEDSGVYFCAR

SEQ ID NO: 16 - protein, 11 aa, synthetic construct
WGQGTTLTVSS

SEQ ID NO: 17 - DNA, 381 bp, synthetic construct
  source 1..381: mol_type = other DNA
atggtatccacacctcagttccttgtatttttgcttttctggattccagcctccagaggtgacatcttgctgactcagtctccagccatcctgtctgtgagtccaggagaaagagtcagcttctcctgcagggccagtcagaacattggcacaagcatacactggtatcagcaaagaacaaatggttttccaaggcttctcataaagtatgcttcggaatctatctctgggatcccttccaggtttagtggcagtggatcagggacagattttactcttagcatcaacagtgtggagtctgaagatattgcagattattactgtcaacaaagttatagctggccattcacgttcggctcggggacaaagttggaaataaaa

SEQ ID NO: 18 - DNA, 33 bp, synthetic construct
  source 1..33: mol_type = other DNA
agggccagtcagaacattggcacaagcatacac

SEQ ID NO: 19 - DNA, 21 bp, synthetic construct
  source 1..21: mol_type = other DNA
tatgcttcggaatctatctct

SEQ ID NO: 20 - DNA, 27 bp, synthetic construct
  source 1..27: mol_type = other DNA
caacaaagttatagctggccattcacg

SEQ ID NO: 21 - DNA, 69 bp, synthetic construct
  source 1..69: mol_type = other DNA
gacatcttgctgactcagtctccagccatcctgtctgtgagtccaggagaaagagtcagcttctcctgc

SEQ ID NO: 22 - DNA, 45 bp, synthetic construct
  source 1..45: mol_type = other DNA
tggtatcagcaaagaacaaatggttttccaaggcttctcataaag

SEQ ID NO: 23 - protein, 96 aa, synthetic construct
GGGATCCCTTCCAGGTTTAGTGGCAGTGGATCAGGGACAGATTTTACTCTTAGCATCAACAGTGTGGAGTCTGAAGATATTGCAGATTATTACTGT

SEQ ID NO: 24 - DNA, 30 bp, synthetic construct
  source 1..30: mol_type = other DNA
ttcggctcggggacaaagttggaaataaaa

SEQ ID NO: 25 - DNA, 402 bp, synthetic construct
  source 1..402: mol_type = other DNA
atggaatggagcggggtctttatctttctcttgtcagtaactgcagatgtccactcccaggtccagttgcagcagtctggagctgagctggtaaggcctgggacttcagtgaagatatcctgtaaggcttctggctacaccttcactaactactggctaggttgggtaaagcagaggcctggacatggacttgagtggattggaaatattttccctggaggtggttatactaactacaatgagaagttcaagggcaaggccatactgactgcagacacatcctccagcactgcctacatgcagctcagtagcctgacatctgaggactctggtgtctatttctgtgcaagagaaggggtcatggactactggggccaaggcaccactctcacagtctcctca

SEQ ID NO: 26 - DNA, 15 bp, synthetic construct
  source 1..15: mol_type = other DNA
aactactggctaggt

SEQ ID NO: 27 - DNA, 51 bp, synthetic construct
  source 1..51: mol_type = other DNA
aatattttccctggaggtggttatactaactacaatgagaagttcaagggc

SEQ ID NO: 28 - DNA, 18 bp, synthetic construct
  source 1..18: mol_type = other DNA
gaaggggtcatggactac

SEQ ID NO: 29 - DNA, 90 bp, synthetic construct
  source 1..90: mol_type = other DNA
caggtccagttgcagcagtctggagctgagctggtaaggcctgggacttcagtgaagatatcctgtaaggcttctggctacaccttcact

SEQ ID NO: 30 - DNA, 42 bp, synthetic construct
  source 1..42: mol_type = other DNA
tgggtaaagcagaggcctggacatggacttgagtggattgga

SEQ ID NO: 31 - DNA, 96 bp, synthetic construct
  source 1..96: mol_type = other DNA
aaggccatactgactgcagacacatcctccagcactgcctacatgcagctcagtagcctgacatctgaggactctggtgtctatttctgtgcaaga

SEQ ID NO: 32 - DNA, 33 bp, synthetic construct
  source 1..33: mol_type = other DNA
tggggccaaggcaccactctcacagtctcctca

SEQ ID NO: 33 - protein, 115 aa, synthetic construct
QVQLVQSGAEVKKPGASVKVSCKASGYTFTNYWLGWVRQAPGQGLEWMGNIFPGGGYTNYNEKFKGRVTMTRDTSTSTVYMELSSLRSEDTAVYYCAREGVMDYWGQGTLVTVSS

SEQ ID NO: 34 - protein, 107 aa, synthetic construct
EIVLTQSPATLSLSPGERATLSCRASQNIGTSIHWYQQKPGQAPRLLIYYASESISGIPARFSGSGSGTDFTLTISSLEPEDFAVYYCQQSYSWPFTFGQGTKLEIK

SEQ ID NO: 35 - DNA, 345 bp, synthetic construct
  source 1..345: mol_type = other DNA
caggttcagctggttcagtctggcgccgaagtgaagaaacctggcgcctctgtgaaggtgtcctgcaaggccagcggctacacctttaccaactactggctcggctgggtccgacaggctcctggacaaggacttgagtggatgggcaacatcttccctggcggcggatacaccaactacaacgagaagttcaagggccgcgtgaccatgaccagagacaccagcacaagcaccgtgtacatggaactgagcagcctgagaagcgaggacaccgccgtgtactactgtgccagagaaggcgtgatggactactggggccagggcacactggtcacagtctcttct

SEQ ID NO: 36 - DNA, 321 bp, synthetic construct
  source 1..321: mol_type = other DNA
gagatcgtgctgacacagtctcccgccacactgtcactgtctccaggcgaaagagccacactgagctgtagagccagccagaacatcggcaccagcatccactggtatcagcagaagcctggacaggcccctcggctgctgatctactatgccagcgagagcatcagcggcatccccgccagattttctggcagcggctctggcaccgatttcaccctgaccataagcagcctggaacctgaggacttcgccgtgtactactgccagcagtcctacagctggcccttcacatttggccagggcaccaagctggaaatcaag

SEQ ID NO: 37 - protein, 110 aa, synthetic construct
APAFSVSPASGLSDGQSVSVSVSGAAAGETYYIAQCAPVGGQDACNPATATSFTTDASGAASFSFVVRKSYTGSTPEGTPVGSVDCATAACNLGAGNSGLDLGHVALTFG

SEQ ID NO: 38 - protein, 9 aa, synthetic construct
GSGGPEGGS

(19) United States
(12) Patent Application Publication
(10) Pub. No.: US 2025/0163176 A1
(43) Pub. Date: May 22, 2025

(54) MONOCLONAL ANTIBODY AGAINST TROP2 AND USE THEREOF

(71) Applicant: INSTITUTE OF MEDICAL BIOTECHNOLOGY, CHINESE ACADEMY OF MEDICAL SCIENCES, Beijing (CN)

(72) Inventors: Qingfang Miao, Beijing (CN); Qun Yu, Beijing (CN); Dandan Zhou, Beijing (CN); Xiaotian Zhai, Beijing (CN); Yongsu Zhen, Beijing (CN)

(21) Appl. No.: 18/966,014

(22) Filed: Dec. 2, 2024

Related U.S. Application Data
(63) Continuation of application No. PCT/CN2023/108534, filed on Jul. 21, 2023.

(30) Foreign Application Priority Data
Jul. 21, 2022 (CN) 202210864288.1

(51) Int. Cl. C07K 16/30; A61K 39/00; A61K 47/68; A61P 35/00; G01N 33/574

(52) U.S. Cl. CPC C07K 16/30; A61K 47/6849; A61P 35/00; G01N 33/57484; A61K 2039/505; C07K 2317/24; C07K 2317/73; C07K 2317/92

(57) ABSTRACT

Provided are an isolated monoclonal antibody or an antigen-binding fragment thereof against Trop2, and a use of the antibody or fragment in tumor treatment and diagnosis.

### TECHNICAL FIELD

The present disclosure relates to the field of biological medicine. Specifically, the present disclosure relates to an isolated monoclonal antibody or an antigen-binding fragment thereof against Trop2, and a use of the antibody or fragment in tumor treatment and diagnosis.

### BACKGROUND

Human trophoblast cell surface antigen 2 (Trop2), also known as tumor-associated calcium signal transducer 2 (TACSTD2), is a transmembrane glycoprotein expressed and encoded by Tacstd2 gene and is a polypeptide formed by 323 amino acids, with a molecular weight of 36 kDa. Trop2 protein, which is first found in human trophoblast cells and described as a surface marker of trophoblast cells, is highly expressed in embryonic progenitor stem cells, promotes the proliferation of embryonic progenitor stem cells and plays a crucial role in tissue and organ formation as well as embryonic development. It is worth noting that Trop2 is also highly expressed in various epithelial malignant tumors, such as oral squamous cell carcinoma, pancreatic cancer, breast cancer, prostate cancer, uterine cancer, ovarian cancer, gastric cancer, colon cancer and lung cancer. The mechanism of Trop2-mediated signaling pathway is not completely clear, which mainly promotes the growth, proliferation, invasion and metastasis of tumor cells by upregulating intracellular calcium concentration, regulating cyclin expression, and reducing fibronectin function. It is found in clinical studies that Trop2 antigen is highly expressed in tumor tissues, and is closely related to the reduction of survival time of patients and poor prognosis. It is shown in immunohistochemical results that anti-Trop2 antibody is highly effective in specific affinity for tumor tissues, making Trop2 a highly promising therapeutic target for tumor-targeted therapies. In recent years, research on antitumor drugs against Trop2, such as antibodies, fusion proteins, chemical inhibitors, nano-preparations, and antibody-drug conjugates (ADC), has been progressively developed, and in particular, research on ADC is developed rapidly. For example, IMMU-132, an ADC in phase II/III clinical trials currently, is formed by coupling a humanized anti-Trop2 monoclonal antibody hRS7 to the topoisomerase inhibitor camptothecin derivative SN-38. This drug shows good antitumor effect and is clinically applied in recurrent and refractory triple-negative breast cancer that has failed to be treated by multiple therapeutic schedules. After administration of this drug, the patients show an objective remission rate of 30% and a clinical effective rate of 46%, with an average time from administration to taking effect of only 1.9 months, greatly improving patient compliance and extending progression-free survival time of the patients to 6 months, with an overall survival time of 16.6 months. In a phase II clinical trial for small cell lung cancer, after being treated with IMMU-132, the patients show an objective remission rate of 19%, a median remission time of 6 months, a clinical effective rate of 43%, a median progression-free survival time of 5.2 months, and a median overall survival time of 9.5 months. Therefore, IMMU-132 brings new hope to patients with triple-negative breast cancer and refractory small cell lung cancer.

Hybridoma fusion technology was first developed by Kohler and Milstein in 1975 and won the Nobel Prize in Physiology or Medicine in 1984. Due to the limited proliferation of B cells and the survival of no more than 20 days in vitro, monoclonal antibodies cannot be produced on a large scale. In contrast, tumor cells can proliferate and survive indefinitely, and therefore, under the action of polyethylene glycol (PEG) fusion agent, B cells capable of producing specific antibodies are fused with myeloma cells (or through electrofusion), and after multiple times of positive clone screening, monoclonal hybridoma cell lines that can stably secrete antibodies against specific antigens are obtained. The antibodies produced are monoclonal antibodies against an antigenic determinant, featuring a high specificity, a high purity, a good homogeneity, a high affinity, a high titer, and a low cost. In recent years, tumor-targeted therapy based on monoclonal antibodies has been recognized as one of the most promising and concerned strategies for tumor treatment. According to the statistics, as of 2018, the Food and Drug Administration (FDA) had approved 24 monoclonal antibody drugs against solid tumors, targeting cluster of differentiation (CD) antigens (including CD19, CD20, CD30, CD33, CD38, and CD53), tumor cell surface molecules (HER2, EGFR, PD-L1, GD2, PMSA, and SLAMF7), immune cell surface inhibitory receptors PD-1 and CTLA-4, and inhibiting tumor angiogenesis. However, at present, target points available for the development of monoclonal antibody drugs are extremely limited. Therefore, it is of great significance to discover new tumor-specific antigens, especially those highly expressed and crucial in tumor tissues, to study and prepare monoclonal antibodies targeting these antigens, and to expand the application of monoclonal antibodies in the field of tumor-targeted therapy.

### SUMMARY

In one aspect, the present disclosure provides a hybridoma cell, deposited at the China General Microbiological Culture Collection Center (CGMCC) under the deposit number CGMCC No. 18167 on Jun. 25, 2019.

In one aspect, the present disclosure provides a monoclonal antibody or an antigen-binding fragment thereof, the monoclonal antibody being produced by a mouse hybridoma cell deposited at CGMCC under the deposit number CGMCC No. 18167 on Jun. 25, 2019.

In one aspect, the present disclosure provides an isolated monoclonal antibody or an antigen-binding fragment thereof against Trop2, the monoclonal antibody containing VL CDR1, VL CDR2, VL CDR3, VH CDR1, VH CDR2, and VH CDR3 of a monoclonal antibody produced by a mouse hybridoma cell deposited at CGMCC under the deposit number CGMCC No. 18167 on Jun. 25, 2019.

In one aspect, the present disclosure provides an isolated monoclonal antibody or an antigen-binding fragment thereof against Trop2, the monoclonal antibody containing a light chain variable region and a heavy chain variable region of a monoclonal antibody produced by a mouse hybridoma cell deposited at CGMCC under the deposit number CGMCC No. 18167 on Jun. 25, 2019.

In one aspect, the present disclosure provides an isolated monoclonal antibody or an antigen-binding fragment thereof against Trop2, the monoclonal antibody containing a light chain variable region and a heavy chain variable region,
the light chain variable region containing:
VL CDR1, containing an amino acid sequence as shown in SEQ ID NO:2 or an amino acid sequence having 1 or 2 amino acid residue substitutions, deletions, or additions relative to SEQ ID NO: 2,
VL CDR2, containing an amino acid sequence as shown in SEQ ID NO:3 or an amino acid sequence having 1 or 2 amino acid residue substitutions, deletions, or additions relative to SEQ ID NO: 3, and
VL CDR3, containing an amino acid sequence as shown in SEQ ID NO:4 or an amino acid sequence having 1 or 2 amino acid residue substitutions, deletions, or additions relative to SEQ ID NO: 4; and
the heavy chain variable region containing:
VH CDR1, containing an amino acid sequence as shown in SEQ ID NO:10 or an amino acid sequence having 1 or 2 amino acid residue substitutions, deletions, or additions relative to SEQ ID NO: 10,
VH CDR2, containing an amino acid sequence as shown in SEQ ID NO:11 or an amino acid sequence having 1 or 2 amino acid residue substitutions, deletions, or additions relative to SEQ ID NO: 11, and
VH CDR3, containing an amino acid sequence as shown in SEQ ID NO:12 or an amino acid sequence having 1 or 2 amino acid residue substitutions, deletions, or additions relative to SEQ ID NO: 12.

In some embodiments, the monoclonal antibody contains a light chain variable region and a heavy chain variable,
the light chain variable region containing:
VL CDR1, containing an amino acid sequence as shown in SEQ ID NO:2,
VL CDR2, containing an amino acid sequence as shown in SEQ ID NO:3, and
VL CDR3, containing an amino acid sequence as shown in SEQ ID NO:4; and
the heavy chain variable region containing:
VH CDR1, containing an amino acid sequence as shown in SEQ ID NO:10,
VH CDR2, containing an amino acid sequence as shown in SEQ ID NO:11, and
VH CDR3, containing an amino acid sequence as shown in SEQ ID NO:12.

In some embodiments, the light chain variable region contains an amino acid sequence as shown in SEQ ID NO:1 or an amino acid sequence having at least 85%, at least 90%, at least 95%, or higher sequence identity to SEQ ID NO:1.

In some embodiments, the heavy chain variable region contains an amino acid sequence as shown in SEQ ID NO:9 or an amino acid sequence having at least 85%, at least 90%, at least 95%, or higher sequence identity to SEQ ID NO:9.

In some implementations, the heavy chain variable region contains an amino acid sequence as shown in SEQ ID NO:33 (variable region of a humanized heavy chain version).

In some implementations, the light chain variable region contains an amino acid sequence as shown in SEQ ID NO:34 (variable region of a humanized light chain version).

In some implementations, the heavy chain variable region contains an amino acid sequence as shown in SEQ ID NO:33, and the light chain variable region contains an amino acid sequence as shown in SEQ ID NO:34.

In another aspect, the present disclosure provides a pharmaceutical composition, containing the monoclonal antibody or an antigen-binding fragment thereof of the present disclosure, and a pharmaceutically acceptable vector.

In some embodiments, the monoclonal antibody or an antigen-binding fragment thereof is conjugated to a therapeutic moiety selected from a cytotoxin, a radioactive isotope, or a biologically active protein.

In another aspect, the present disclosure provides a method for treating and/or preventing Trop2-related diseases in patients, which involves administering to the patients an effective amount of the monoclonal antibody or an antigen-binding fragment thereof of the present disclosure, or the pharmaceutical composition of the present disclosure.

In some embodiments, the Trop2-related diseases are malignant tumors highly expressing Trop2. In some embodiments, the malignant tumors are epithelial malignant tumors. In some embodiments, the malignant tumor is selected from male/female reproductive system tumors (such as endometrial cancer, uterine cancer, cervical cancer, breast cancer, ovarian cancer and prostate cancer), digestive system tumors (such as pancreatic cancer, colon cancer, gastric cancer, esophageal squamous cell carcinoma, esophageal cancer, cholangiocarcinoma and intestinal cancer), head and neck tumors (such as oral squamous cell carcinoma and throat cancer), nervous system tumors (such as brain glioma), and respiratory system tumors (such as lung cancer, for example, small cell lung cancer).

In some embodiments, the method also includes applying additional antitumor therapeutic means to the patients, such as applying a chemotherapeutic agent, an antibody targeting other tumor-specific antigens, or radiotherapy.

In another aspect, the present disclosure provides a use of the monoclonal antibody or an antigen-binding fragment thereof of the present disclosure, or the pharmaceutical composition of the present disclosure in preparing a drug for treating and/or preventing Trop2-related diseases.

In some embodiments, the Trop2-related diseases are malignant tumors highly expressing Trop2. In some embodiments, the malignant tumors are epithelial malignant tumors. In some embodiments, the malignant tumor is selected from male/female reproductive system tumors (such as endometrial cancer, uterine cancer, cervical cancer, breast cancer, ovarian cancer and prostate cancer), digestive system tumors (such as pancreatic cancer, colon cancer, gastric cancer, esophageal squamous cell carcinoma, esophageal cancer, cholangiocarcinoma and intestinal cancer), head and neck tumors (such as oral squamous cell carcinoma and throat cancer), nervous system tumors (such as brain glioma), and respiratory system tumors (such as lung cancer, for example, small cell lung cancer).

In another aspect, the present disclosure also provides a method for detecting the presence or expression level of Trop2 in a biological sample, including contacting the biological sample and a control sample with the monoclonal antibody or an antigen-binding fragment thereof against Trop2 of the present disclosure under conditions where a composition can form between the monoclonal antibody or an antigen-binding fragment thereof against Trop2 of the present disclosure and Trop2; and detecting the formation of the composition. The difference in the composition formation between the biological sample and the control sample indicates the presence or expression level of Trop2 in the sample.

In another aspect, the present disclosure also provides a method for detecting the presence of malignant tumors in patients, including:
a) contacting a biological sample obtained from the patient with the monoclonal antibody or an antigen-binding fragment thereof of the present disclosure; and
b) detecting the binding of the monoclonal antibody or an antigen-binding fragment thereof to a target antigen in the biological sample, the detected binding indicating the presence of malignant tumors in the patient.

In some embodiments of the foregoing aspects, the biological sample includes a blood sample, a lymphatic sample, or a component thereof. In some embodiments, the malignant tumor is selected from male/female reproductive system tumors (such as endometrial cancer, uterine cancer, cervical cancer, breast cancer, ovarian cancer and prostate cancer), digestive system tumors (such as pancreatic cancer, colon cancer, gastric cancer, esophageal squamous cell carcinoma, esophageal cancer, cholangiocarcinoma and intestinal cancer), head and neck tumors (such as oral squamous cell carcinoma and throat cancer), nervous system tumors (such as brain glioma), and respiratory system tumors (such as lung cancer, for example, small cell lung cancer).

In another aspect, the present disclosure provides a diagnostic agent for detecting and/or diagnosing Trop2-related diseases such as malignant tumors, containing the monoclonal antibody or an antigen-binding fragment thereof against Trop2 of the present disclosure, and an optionally physiologically acceptable vector.

In another aspect, the present disclosure provides a use of the monoclonal antibody or an antigen-binding fragment thereof against Trop2 of the present disclosure in preparing a diagnostic agent for detecting and/or diagnosing Trop2-related diseases such as malignant tumors.

In another aspect, the present disclosure provides a method for detecting and/or diagnosing Trop2-related diseases such as malignant tumors in a subject, including administering to the subject the monoclonal antibody or an antigen-binding fragment thereof against Trop2 of the present disclosure, or the diagnostic agent of the present disclosure.

In another aspect, the present disclosure also provides an isolated nucleic acid molecule, encoding the monoclonal antibody or an antigen-binding fragment thereof of the present disclosure.

In some embodiments, the nucleic acid molecule encodes complementary determining regions (CDRs) of a light chain variable region and a heavy chain variable region of the antibody or an antigen-binding fragment thereof of the present disclosure. Specifically, the nucleic acid molecule has nucleotide sequences as shown in SEQ ID NO:18-20 or 26-28.

In some embodiments, the nucleic acid molecule encodes a light chain variable region or a heavy chain variable region of the antibody or an antigen-binding fragment thereof of the present disclosure. In some embodiments, the nucleic acid molecule has a nucleotide sequence as shown in SEQ ID NO:17 or 25. In some implementations, the nucleic acid molecule has nucleotide sequences as shown in SEQ ID NO:35-36.

In some embodiments, the nucleic acid molecule is operatively linked to an expression regulatory sequence.

In another aspect, the present disclosure also provides an expression vector, containing the nucleic acid molecule of the present disclosure.

In another aspect, the present disclosure also provides a host cell, transformed with the nucleic acid molecule of the present disclosure or the expression vector of the present disclosure.

In one aspect, the present disclosure also provides a method for producing the monoclonal antibody or an antigen-binding fragment thereof against Trop2: including:
(i) culturing the host cell of the present disclosure under conditions suitable for expression of the nucleic acid molecule or the expression vector of the present disclosure, and
(ii) isolating and purifying an antibody or an antigen-binding fragment thereof expressed by the nucleic acid molecule or the expression vector.

### BRIEF DESCRIPTION OF THE DRAWINGS

FIG. 1 shows the identification of chromosome number in a hybridoma cell, with an average chromosome number of 104, which is consistent with the theoretical value for hybridoma cell chromosomes, and with the presence of characteristic chromosomes of hybridoma (marked with circles).

FIG. 2 shows the titer of an antibody detected by enzyme-linked immunosorbent assay (ELISA), with the titer as high as 8000000.

FIG. 3 shows the sodium dodecyl sulfate-polyacrylamide gel electrophoresis (SDS-PAGE) diagram of the antibody (Lane M: Marker, Lane 1: IMB1636, Lane 2: VH and VL).

FIG. 4 shows the purity of the antibody detected by the high performance liquid chromatography (HPLC), with the purity up to 99%.

FIG. 5 shows the identification of antibody subtype, and the result determines that the subclass of the antibody is IgG1 and the monoclonal antibody light chain is Kappa.

FIG. 6 shows the antibody-antigen affinity analysis conducted using the Biacore method, with ka (1/Ms)=1.057×10⁶, kd (1/s)=7.297×10⁻⁴, and KD=4.842×10⁻¹⁰.

FIG. 7 shows antibody-antigen affinity reaction curves obtained by ELISA, obtaining the affinity constant Ka=3.9×10¹⁰.

FIG. 8 is a diagram showing the binding ability of the antibody to tumor cells by immunofluorescence analysis, proving that the antibody has high membrane-binding activity with the tumor cell membrane BxPC-3 that highly expresses Trop2.

FIG. 9 shows the analysis of antibody-tumor cell affinity activity based on confocal microscopy, proving that Trop2-positive cells HCC-827 can efficiently endocytose the antibody.

FIG. 10 is a diagram demonstrating the binding ability of the antibody to tumor cells by flow cytometry, proving that the antibody has high affinity activity with the tumor cell membrane that highly expresses Trop2 antigen.

FIG. 11 shows in vivo imaging detection of mice, showing that the antibody can highly target tumor tissues in vivo and remain in the tumor tissue for over 11 days.

FIG. 12 shows human tumor tissue chip detection, showing that the antibody can highly and specifically bind to human lung squamous cell carcinoma tumor tissues.

FIG. 13 shows the amino acid sequence information of a mouse-derived anti-Trop2 antibody.

FIG. 14 shows the nucleic acid sequence information of the mouse-derived anti-Trop2 antibody.

FIG. 15 shows the amino acid sequence information of a humanized anti-Trop2 antibody.

FIG. 16 shows the nucleic acid sequence information of the humanized anti-Trop2 antibody.

FIG. 17 shows the antitumor effect of Anti-Trop2-lidamycin (LDM) in vivo.

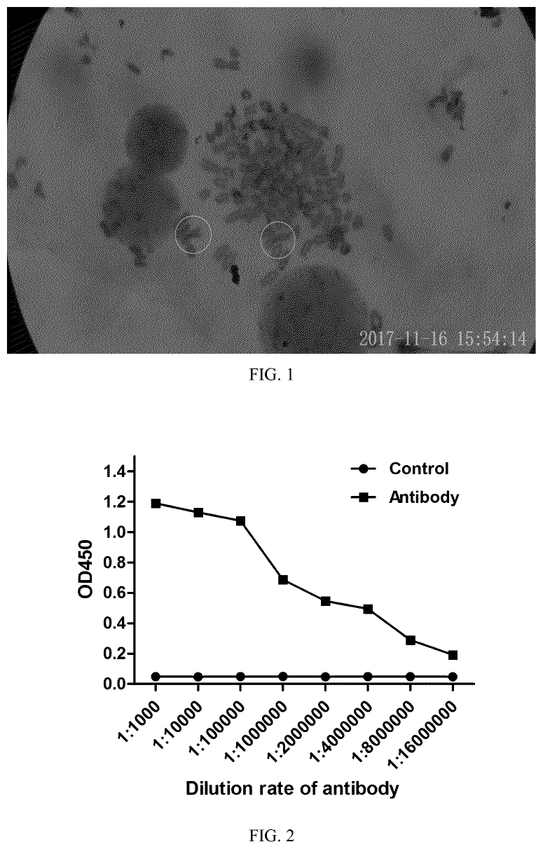

### DETAILED DESCRIPTION

### I. Definition

In the present disclosure, unless otherwise defined, all the scientific and technical terms used herein have the same meaning as those generally understood by those skilled in the technical field to which the present disclosure belongs. Additionally, the terms and laboratory operation procedures related to protein and nucleic acid chemistry, molecular biology, cell and tissue culture, microbiology, and immunology used herein are those widely used in the corresponding fields. Meanwhile, to better understand the present disclosure, the definition and explanation of relevant terms are provided below.

As used herein, the term “antibody” refers to immunoglobulins and immunoglobulin fragments, whether produced naturally, or synthesized (recombined) partially or wholly, including any fragments that contain at least a part of the variable region of the immunoglobulin molecule and retain the binding specificity of the full-length immunoglobulin. Therefore, the antibody includes any proteins having a binding structure domain that is homologous or essentially homologous to an immunoglobulin antigen-binding structure domain (antibody binding site). The antibody includes antibody fragments, such as antitumor cell antibody fragments. As used herein, the term “antibody” therefore includes synthetic antibodies, recombinant antibodies, multi-specific antibodies (such as bispecific antibodies), human antibodies, non-human antibodies, humanized antibodies, chimeric antibodies, intracellular antibodies, and antibody fragments including but not limited to, fragments, Fab′ fragments, F(ab′)₂ fragments, Fv fragments, disulfide-linked Fv (dsFv), Fd fragments, Fd′ fragments, single-chain Fv (scFv), single-chain Fab (scFab), bis-antibodies, anti-idiotype (anti-Id) antibodies, or antigen-binding fragments of any one of the foregoing antibodies. The antibody provided herein includes members of any immunoglobulin types (such as IgG, IgM, IgD, IgE, IgA, and IgY), any classes (such as IgG1, IgG2, IgG3, IgG4, IgA1, and IgA2), or subclasses (such as IgG2a and IgG2b).

As used herein, the term “antibody fragment” or “antigen-binding fragment” of an antibody refers to any parts of a full-length antibody, less than full-length but containing at least a part of the variable region of the antibody that binds to an antigen (such as one or more CDRs and/or one or more antibody-binding sites), and thus retaining the binding specificity and at least part of the binding specificity of the full-length antibody. Therefore, the antigen-binding fragment refers to an antibody fragment containing an antigen-binding part that binds to the same antigen as the antibody from which the antibody fragment is derived. The antibody fragment includes antibody derivatives derived from enzymatically treated full-length antibody, and derivatives produced via synthesis, such as derivatives produced via recombination. The antibody includes antibody fragments. Examples of the antibody fragment include, but are not limited to, Fab, Fab′, F(ab′)₂, scFv, Fv, dsFv, bis-antibodies, Fd and Fd′ fragments, and other fragments including the modified fragments (seen in, e.g., Methods in Molecular Biology, Vol 207: Recombinant Antibodies for Cancer Therapy Methods and Protocols (2003); Chapter 1; p 3-25, Kipriyanov). The fragment can include a plurality of chains linked together, such as, by disulfide bonds and/or by peptide linkers. The antibody fragment generally contains at least or about 50 amino acids, and typically contains at least or about 200 amino acids. The antigen-binding fragment includes any antibody fragments that, when inserted into an antibody framework (for instance, through the replacement of corresponding regions), acquire an antibody capable of immunologically and specifically binding (i.e., exhibiting at least or at least about 10⁷-10⁸ M⁻¹ of Ka) to an antigen.

As used herein, the term “monoclonal antibody” refers to a population having identical antibody, representing that each individual antibody molecule in the monoclonal population of the antibody is the same as the others. This characteristic is opposite to that of a polyclonal population of the antibody, which contains antibodies with various sequences. The monoclonal antibody can be prepared through many well-known methods (seen in: Smith et al. (2004) J. Clin. Pathol. 57, 912-917; and Nelson et al., J Clin Pathol (2000), 53, 111-117). For example, the monoclonal antibody can be prepared by immortalizing B cells, which involves, such as, fusion of B cells with myeloma cells to produce hybridoma cell lines or infection of B cells with viruses such as Epstein-Barr virus (EBV). Recombinant techniques can also be employed to prepare antibodies from cloned populations of host cells in vitro, involving transforming the host cells with plasmids carrying artificial sequences of nucleotides encoding the antibody.

As used herein, the term “hybridoma” or “hybridoma cell” refers to a cell or cell line (typically a myeloma or lymphoma cell) produced by fusing antibody-producing lymphocytes and non-antibody-producing cancer. As is known to those ordinary skilled in the field, the hybridoma can proliferate and continuously produce specific monoclonal antibodies. The method for producing the hybridoma is known in the field (seen in, for example, Harlow & Lane, 1988). The term “hybridoma” or “hybridoma cell” also includes subclones and progeny cells of the hybridoma.

As used herein, the term “conventional antibody” refers to an antibody containing two heavy chains (which can be represented as H and H′), two light chains (which can be represented as L and L′), and two antigen-binding sites. Each heavy chain can be a full-length immunoglobulin heavy chain or any functional regions thereof retaining antigen-binding ability (for example, the heavy chain includes but is not limited to V_{H} chains, V_{H}-C_{H}1 chains, and V_{H}-C_{H}1-C_{H}2-C_{H}3 chains), and each light chain can be a full-length light chain or any functional regions thereof (for example, the light chain includes but is not limited to V_{L} chains and V_{L}-C_{L} chains). Each heavy chain (H and H′) pairs with one light chain (L and L′).

As used herein, a full-length antibody is an antibody having two full-length heavy chains (such as V_{H}-C_{H}1-C_{H}2-C_{H}3 or V_{H}-C_{H}1-C_{H}2-C_{H}3-C_{H}4), two full-length light chains (V_{L}-C_{L}), and a hinge region, such as antibodies naturally produced by antibody-secreting B cells or antibodies synthetically produced with identical structure domains.

As used herein, dsFv refers to an engineered intermolecular disulfide-bonded Fv with stable V_{H}-V_{L} pairs.

As used herein, a Fab fragment refers to an antibody fragment obtained by papain digestion of a full-length immunoglobulin, or a fragment having the same structure, for example, produced by synthesis using recombinant method. The Fab fragment contains light chains (including V_{L} and C_{L}) and another chain containing a variable domain of the heavy chain (V_{H}) and one constant region structure domain (C_{H}1) of the heavy chain.

As used herein, a F(ab′)₂ fragment refers to an antibody fragment obtained by pepsin digestion of an immunoglobulin under pH 4.0-4.5, or a fragment having the same structure, for example, produced by synthesis using recombinant method. The F(ab′)₂ fragment essentially contains two Fab fragments, with each heavy chain part containing additional amino acids including cysteines that form disulfide bonds connecting two fragments.

As used herein, a Fab′ fragment is a fragment containing half of an F(ab′)₂ fragment (one heavy chain and one light chain).

As used herein, a scFv fragment refers to an antibody fragment containing covalently linked variable light chain (V_{L}) and variable heavy chain (V_{H}) in any order through a polypeptide linker. The length of the linker enables two variable structure domains to be bridged basically without interference. An exemplary linker is a (Gly-Ser)ₙ residue interspersed with some Glu or Lys residues to increase solubility.

The term “chimeric antibody” refers to an antibody that has a variable region sequence derived from one species and a constant region sequence derived from another, such as, an antibody with a variable region sequence derived from a mouse antibody and a constant region sequence derived from a human antibody.

A “humanized” antibody refers to an antibody in non-human (such as mouse) antibody, which is a chimeric immunoglobulin, immunoglobulin chain, or fragment thereof (such as Fv, Fab, Fab′, F(ab′)₂, or other antigen-binding subsequence of an antibody), containing minimal sequences derived from a non-human immunoglobulin. Preferably, the humanized antibody is a human immunoglobulin (acceptor antibody), and residues from CDR of the acceptor antibody are replaced with CDR residues from a non-human species (donor antibody) with desired specificity, affinity, and capability, such as a mouse, rat, or rabbit.

Furthermore, in humanization, amino acid residues in CDR1, CDR2, and/or CDR3 of VH and/or VL may be mutated to improve one or more binding characteristics (such as affinity) of the antibody. For example, polymerase chain reaction (PCR)-mediated mutations can be introduced, the effect of which on antibody binding or other functional properties can be assessed using in vitro or in vivo tests described herein. Typically, conservative mutations are introduced, such as amino acid substitutions, additions, or deletions. Additionally, mutations in CDR are usually no more than one or two. Thus, the humanized antibody of the present disclosure also includes antibodies having 1 or 2 amino acid mutations in CDR.

As used herein, the term “epitope” refers to any antigenic determinants on an antigen that binds to the complementary site of an antibody. An epitope determinant usually contains a chemically active surface subtype of a molecule, such as an amino acid or sugar side chain, and usually has specific three-dimensional structural characteristics and specific charge characteristics.

As used herein, a variable structure domain or variable region is a specific Ig structure domain of a heavy chain or light chain of an antibody, containing amino acid sequences varying between different antibodies. Each light chain and each heavy chain has one variable structure domain V_{L} and V_{H}, respectively. The variable structure domain provides antigen specificity and is thus responsible for antigen recognition. Each variable region contains CDR and framework region (FR), with CDR being a part of an antigen-binding site structure domain.

As used herein, “antigen-binding structure domain” and “antigen-binding site” synonymously refer to a structure domain in an antibody that recognizes and physically interacts with a cognate antigen. A natural conventional full-length antibody molecule has two conventional antigen-binding sites, each containing a heavy chain variable region part and a light chain variable region part. The conventional antigen-binding site contains a loop connecting antiparallel β-strands in the variable structure domain. The antigen-binding site may contain other parts of the variable structure domain. Each conventional antigen-binding site contains three hypervariable regions from the heavy chain and three hypervariable regions from the light chain. Hypervariable regions are also known as CDR.

As used herein, “hypervariable region”, “HV”, “CDR”, and “antibody CDR” can be used interchangeably to refer to one of the multiple parts within various variable structure domains that together form the antigen-binding site of an antibody. Each variable region structure domain contains three CDRs, named CDR1, CDR2, and CDR3. For example, a light chain variable region structure domain contains three CDRs, named VL CDR1, VL CDR2, and VL CDR3; a heavy chain variable region structure domain contains three CDRs, named VH CDR1, VH CDR2, and VH CDR3. The three CDRs in the variable region are discontinuous along the linear amino acid sequence but are close in the folded polypeptide. CDRs are located within a loop connecting β-folded parallel strands of the variable domain. As described herein, CDR can be known and identified by those skilled in the field based on Kabat or Chothia numbering (seen in, for example, Kabat, E. A. et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917).

As used herein, FR is a structure domain within a variable region structure domain of an antibody that located in β-strands; and FR is relatively more conserved than hypervariable region in terms of amino acid sequence.

As used herein, a “constant region” structure domain refers to a structure domain in a heavy chain or light chain of an antibody, containing an amino acid sequence relatively more conserved than that in the variable region structure domain. In conventional full-length antibody molecules, each light chain has a single light chain constant region (C_{L}) structure domain, and each heavy chain contains one or more heavy chain constant region (C_{H}) structure domains, including C_{H}1, C_{H}2, C_{H}3, and C_{H}4. Full-length IgA, IgD, and IgG isotypes contain C_{H}1, C_{H}2, C_{H}3, and a hinge region. IgE and IgM contain C_{H}1, C_{H}2, C_{H}3, and C_{H}4. The C_{H}1 and C_{L} structure domains serve for extending Fab arms of the antibody molecule, contributing to interaction with antigen and rotating antibody arms. The constant region of an antibody can serve effector functions, such as but not limited to clearing antigens, pathogens, and toxins specifically bound to the antibody, for example, through interactions with various cells, biomolecules, and tissues.

As used herein, a functional region of an antibody is an antibody part that contains at least V_{H}, V_{L}, C_{H} (such as C_{H}1, C_{H}2, or C_{H}3), C_{L}, or a hinge region structure domain or at least a functional region thereof.

As used herein, a functional region of a V_{H} structure domain is at least a part of an intact V_{H} structure domain that retains at least part of the binding specificity of the intact V_{H} structure domain (e.g., by retaining one or more CDRs of the intact V_{H} structure domain), and therefore the functional region of the V_{H} structure domain binds to an antigen alone, or in combination with another antibody structure domain (such as a V_{L} structure domain) or a region thereof. An exemplary functional region of a V_{H} structure domain is a region containing CDR1, CDR2, and/or CDR3 of the V_{H} structure domain.

As used herein, a functional region of a V_{L} structure domain refers to at least a part of an intact V_{L} structure domain that retains at least part of binding specificity of the intact V_{L} structure domain (for example, by retaining one or more CDRs of the intact V_{L} structure domain), and therefore the functional region of the V_{L} structure domain binds to an antigen alone, or in combination with another antibody structure domain (such as a V_{H} structure domain) or a region thereof. An exemplary functional region of a V_{L} structure domain is a region containing CDR1, CDR2, and/or CDR3 of the V_{L} structure domain.

As used herein, the term “specific binding” or “immunospecific binding” about an antibody or an antigen-binding fragment thereof can be interchangeably used herein, and refers to the ability of the antibody or antigen-binding fragment to form one or more non-covalent bonds with the same antigen via non-covalent interactions between the antibody-binding site of the antibody and the antigen. The antigen can be an isolated antigen or an antigen present in tumor cells. Typically, an antibody that immunospecifically binds (or specifically binds) to an antigen does so with an affinity constant Ka of about or 1×10⁷ M⁻¹ or 1×10⁸ M⁻¹ or greater (or with a dissociation constant Kd of 1×10⁻⁷ M or 1×10⁻⁸ M or lower). The affinity constant can be determined by standard kinetic methods for antibody reactions, such as immunoassay, surface plasmon resonance (SPR) (Rich and Myszka (2000) Curr. Opin. Biotechnol 11:54; Englebienne (1998) Analyst 123:1599), isothermal titration calorimetry (ITC), or other kinetic interaction assays known in the art (seen in, for example, Paul, ed., Fundamental Immunology, 2nd ed., Raven Press, New York, pages 332-336 (1989); also seen in U.S. Pat. No. 7,229,619 describing exemplary SPR and ITC methods for calculating binding affinity of antibodies). Instruments and methods for real-time detection and monitoring of binding rates are known and commercially available (seen in, BiaCore 2000, Biacore AB, Upsala, Sweden, and GE Healthcare Life Sciences; Malmqvist (2000) Biochem. Soc. Trans. 27:335).

As used herein, the term “compete” about antibodies refers to a first antibody or an antigen-binding fragment thereof binding to an epitope in a manner sufficiently similar to that of a second antibody or an antigen-binding fragment thereof, and therefore, the binding of the first antibody to an associated epitope thereof is detectably reduced in the presence of the second antibody compared to the absence of the second antibody. Or, it can be, but is not necessarily, the case where the binding of the second antibody to an epitope thereof is detectably reduced in the presence of the first antibody. That is, the first antibody can inhibit the binding of the second antibody to the epitope thereof without the second antibody inhibiting the binding of the first antibody to a respective epitope thereof. However, in a case where each antibody can detectably inhibit the binding of the other to an associated epitope thereof or a ligand, whether in the same, greater, or lesser degree, the antibodies are known as “cross-competing” for binding to their respective epitopes. The antibodies involve competing and cross-competing are included in the present disclosure. Regardless of the mechanism by which such competition or cross-competition occurs (such as steric hindrance, conformational change, or binding to a common epitope or fragment thereof), those skilled in the field, based on the instructions provided by the present disclosure, will recognize that such antibodies involving competing and/or cross-competing are included in the present disclosure and can be used in the method disclosed in the present disclosure.

As used herein, the term “polypeptide” refers to two or more amino acids covalently linked. The terms “polypeptide” and “protein” can be interchangeably used herein.

The term “isolated protein”, “isolated polypeptide”, or “isolated antibody” refers to a fact that a protein, polypeptide, or antibody (1) is not associated with naturally associated components thereof in a native state, (2) contains no other proteins from the same species, (3) is expressed by cells from a different species, or (4) does not occur naturally. Therefore, a chemically synthesized polypeptide or a polypeptide synthesized in a cell system different from the natural source cell of the polypeptide will be “isolated” from its naturally associated components. The protein can also be isolated to be substantially free of naturally associated components using protein purification techniques that is well-known in the field.

In peptides or proteins, suitable conservative amino acid substitutions are known to those skilled in the field and can generally be conducted without altering the biological activity of the obtained molecule. Generally, those skilled in the field recognize that a single amino acid substitution in a non-essential region of a polypeptide substantially involves no change in biological activity (seen in, for example, Watson et al., Molecular Biology of the Gene, 4th Edition, 1987, The Benjamin/Cummings Pub. Co., p. 224).

As used herein, the terms “polynucleotide” and “nucleic acid molecule” refer to oligomers or polymers containing at least two linked nucleotides or nucleotide derivatives, including deoxyribonucleic acid (DNA) and ribonucleic acid (RNA) that are typically linked together by phosphodiester bonds.

As used herein, the isolated nucleic acid molecule is a nucleic acid molecule isolated from other nucleic acid molecules naturally present in the nucleic acid molecule. For example, an “isolated” nucleic acid molecule of a complementary deoxyribonucleic acid (cDNA) molecule can be essentially free of other cellular material or medium when prepared by recombinant techniques, or essentially free of chemical precursors or other chemical components when chemically synthesized. Exemplary isolated nucleic acid molecules provided herein include isolated nucleic acid molecules encoding the provided antibody or antigen-binding fragment.

The term sequence “identity” has the meaning generally recognized in the field, and the percentage of sequence identity between two nucleic acids or polypeptide molecules or regions can be calculated using publicly available techniques. Sequence identity can be measured along the full length of a polynucleotide or polypeptide, or along a region of the molecule (seen in, for example: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Although there are many methods for measuring identity between two polynucleotides or polypeptides, the term “identity” is well-known to those skilled in the field (Carrillo, H. & Lipman, D., SIAM J Applied Math 48:1073 (1988)).

As used herein, “operatively linked” about a nucleic acid sequence, region, element or structure domain indicates that the nucleic acid regions are functionally related to each other. For example, a promoter can be operatively linked to a nucleic acid encoding a polypeptide, and therefore the promoter regulates or mediates the transcription of the nucleic acid.

As used herein, the term “expression” refers to the process of generating a polypeptide through transcription and translation of a polynucleotide. The expression level of the polypeptide can be evaluated using any method known in the field, including methods for determining the amount of polypeptide produced from a host cell. Such methods can include, but are not limited to, quantification of peptides in cell lysates by ELISA, gel electrophoresis followed by Coomassi blue staining, Lowry protein assay, and Bradford protein assay.

As used herein, the term “host cell” refers to a cell used to receive, retain, replicate and amplify a vector. The host cell can also be used to express a polypeptide encoded by the vector. When the host cell divides, the nucleic acid contained in the vector is replicated, thereby amplifying the nucleic acid. The host cell can be a eukaryotic or prokaryotic cell. Suitable host cells include, but are not limited to, Chinese hamster ovary (CHO) cells, various COS cells, HeLa cells, human embryonic kidney (HEK) cells such as HEK 293 cells.

“Codon optimization” refers to a method for modifying a nucleic acid sequence to enhance expression in a host cell of interest, which is achieved by replacing at least one codon (e.g., about 1, 2, 3, 4, 5, 10, 15, 20, 25, 50 or more codons) of a natural sequence with a codon that is more frequently or most frequently used in the gene of the host cell while maintaining the natural amino acid sequence. Different species exhibit specific preferences for certain codons of specific amino acids. Codon preference (differences in codon usage between organisms) is often associated with the translation efficiency of messenger ribonucleic acid (mRNA), and this translation efficiency is regarded as to be dependent on the nature of the translated codon and the availability of specific transfer RNA (tRNA) molecules. The advantage of selected tRNA in the cell generally reflects the codon most frequently used for peptide synthesis. Thus, genes can be customized for optimizing gene expression in a given organism based on codon. Tables of codon utilization rate are easily available. For example, a Codon Usage Database can be available on www.kazusa.orip/codon/. These tables can be adjusted and adapted in different ways (seen in, Nakamura Y. et al, “Codon usage tabulated from the international DNA sequence databases: status for the year 2000. Nucl. Acids Res., 28:292 (2000)).

As used herein, the term “vector” is a replicable nucleic acid. When the vector is transformed into a suitable host cell, one or more heterologous proteins can be expressed from the vector. The vector includes vectors, into which, nucleic acids encoding polypeptides or fragments thereof can be introduced generally by restricting enzyme digestion and ligation. The vector also includes vectors containing nucleic acids encoding polypeptide. The vector serves to introduce nucleic acids encoding polypeptide into host cell for amplification of the nucleic acids or for expression/display of the polypeptide encoded by the nucleic acids. The vector generally remains free, but can be designed as a chromosome to integrate the gene or a part thereof into the genome. Vectors of artificial chromosomes are also included, such as vectors of yeast artificial chromosomes and mammalian artificial chromosomes. The selection and use of such vectors are well known to those skilled in the field.

As used herein, the vector also includes a “viral vector” or “vector of a virus”. The vector of a virus is an engineered virus that is operatively linked to an exogenous gene to transfer the exogenous gene (as a vehicle or shuttle) into cells.

As used herein, the term “expression vector” includes vectors capable of expressing DNA, and the DNA is operatively linked to regulatory sequences of promoter regions that can influence the expression of such DNA fragments. Such additional fragments can include promoter and terminator sequences, and optionally can include one or more origins of replication, one or more selectable markers, enhancers, polyadenylation signals, etc. The expression vector is generally derived from plasmids or viral DNA, or can contain elements of the two. Thus, the expression vector refers to recombinant DNA or RNA constructs, such as plasmids, phages, recombinant viruses, or other vectors. When the expression vector is introduced into a suitable host cell, the expression of cloned DNA is obtained. The suitable expression vector is well known to those skilled in the field and includes replicable expression vectors in eukaryotic and/or prokaryotic cells, as well as expression vectors that remain free or integrate into the host cell genome.

As used herein, the “treatment” of an individual with diseases or disease conditions represents that the symptoms of the individual are alleviated partially or completely or maintain unchanged after treatment. Therefore, the treatment includes prevention, therapy, and/or cure. The prevention refers to preventing a potential disease and/or preventing symptoms from worsening or diseases from developing. The treatment also includes any pharmaceutical use of any antibody or antigen-binding fragment thereof, as well as composition provided herein.

As used herein, “therapeutic effect” refers to the effect obtained by the treatment of an individual, altering, typically improving, or ameliorating symptoms of a disease or disease condition or curing the disease or disease condition.

As used herein, a “therapeutically effective amount” or “therapeutically effective dosage” refers to an amount of a substance, compound, material, or composition containing a compound that is enough, after administration to a subject, to at least produce a therapeutic effect. Therefore, it is the amount necessary to prevent, cure, ameliorate, arrest, or partially arrest the symptoms of a disease or disorder.

As used herein, a “prophylactically effective amount” or “prophylactically effective dosage” refers to an amount of a substance, compound, material, or composition containing a compound that, when administered to a subject, will have the desired prophylactic effect, such as preventing or delaying the occurrence or recurrence of a disease or symptoms, and reducing the possible occurrence or recurrence of a disease or symptoms. A fully prophylactic effective dosage does not necessarily to be achieved through the administration of one dosage and may be achieved after a series of dosages. Therefore, the prophylactically effective amount can be applied in one or multiple administrations.

As used herein, the term “patients” refers to mammals, such as humans.

### II. Monoclonal Antibody Against Trop2

In the present disclosure, by means of hybridoma fusion technology, immune spleen cells are prepared by immunizing BALB/C mice with Trop2 antigen, and are then fused with SP2/0 cells, to establish a library of hybridoma cell line that secretes anti-Trop2 monoclonal antibodies, from which, a hybridoma cell line Y1636-1 that stably and highly secretes antibodies is screened. The ascites induction method is employed to expand the production of monoclonal antibodies, thereby preparing a mouse-derived anti-Trop2 monoclonal antibody with a high titer, high affinity, and high specificity. An amino acid sequence of the antibody is determined, and the mouse-derived monoclonal antibody is humanized. The mouse hybridoma cell line Y1636-1 that produces anti-Trop2 monoclonal antibodies was deposited at CGMCC under the deposit number CGMCC No. 18167 on Jun. 25, 2019.

Therefore, in one aspect, the present disclosure provides a hybridoma cell, deposited at CGMCC under the deposit number CGMCC No. 18167 on Jun. 25, 2019.

In one aspect, the present disclosure provides a monoclonal antibody or an antigen-binding fragment thereof, the monoclonal antibody being produced by a mouse hybridoma cell deposited at CGMCC under the deposit number CGMCC No. 18167 on Jun. 25, 2019.

In one aspect, the present disclosure provides an isolated monoclonal antibody or an antigen-binding fragment thereof against Trop2, the monoclonal antibody containing VL CDR1, VL CDR2, VL CDR3, VH CDR1, VH CDR2, and VH CDR3 of a monoclonal antibody produced by a mouse hybridoma cell deposited at CGMCC under the deposit number CGMCC No. 18167 on Jun. 25, 2019.

In one aspect, the present disclosure provides an isolated monoclonal antibody or an antigen-binding fragment thereof against Trop2, the monoclonal antibody containing a light chain variable region and a heavy chain variable region of a monoclonal antibody produced by a mouse hybridoma cell deposited at CGMCC under the deposit number CGMCC No. 18167 on Jun. 25, 2019.

Therefore, the present disclosure provides an isolated monoclonal antibody or an antigen-binding fragment thereof against Trop2, the monoclonal antibody containing a light chain variable region and a heavy chain variable region.

The light chain variable region contains:
VL CDR1, containing an amino acid sequence as shown in SEQ ID NO:2 or an amino acid sequence having 1 or 2 amino acid residue substitutions, deletions, or additions relative to SEQ ID NO: 2,
VL CDR2, containing an amino acid sequence as shown in SEQ ID NO:3 or an amino acid sequence having 1 or 2 amino acid residue substitutions, deletions, or additions relative to SEQ ID NO: 3, and
VL CDR3, containing an amino acid sequence as shown in SEQ ID NO:4 or an amino acid sequence having 1 or 2 amino acid residue substitutions, deletions, or additions relative to SEQ ID NO: 4; and
the heavy chain variable region contains:
VH CDR1, containing an amino acid sequence as shown in SEQ ID NO:10 or an amino acid sequence having 1 or 2 amino acid residue substitutions, deletions, or additions relative to SEQ ID NO: 10,
VH CDR2, containing an amino acid sequence as shown in SEQ ID NO:11 or an amino acid sequence having 1 or 2 amino acid residue substitutions, deletions, or additions relative to SEQ ID NO: 11, and
VH CDR3, containing an amino acid sequence as shown in SEQ ID NO:12 or an amino acid sequence having 1 or 2 amino acid residue substitutions, deletions, or additions relative to SEQ ID NO: 12.

In some embodiments, the monoclonal antibody contains a light chain variable region and a heavy chain variable.

The light chain variable region contains:
VL CDR1, containing an amino acid sequence as shown in SEQ ID NO:2,
VL CDR2, containing an amino acid sequence as shown in SEQ ID NO:3, and
VL CDR3, containing an amino acid sequence as shown in SEQ ID NO:4; and
the heavy chain variable region contains:
VH CDR1, containing an amino acid sequence as shown in SEQ ID NO:10,
VH CDR2, containing an amino acid sequence as shown in SEQ ID NO:11, and
VH CDR3, containing an amino acid sequence as shown in SEQ ID NO:12.

In some implementations, the monoclonal antibody is a humanized antibody.

In some implementations, the light chain variable region contains an amino acid sequence as shown in SEQ ID NO:1 or an amino acid sequence having at least 85%, at least 90%, at least 95%, or higher sequence identity to SEQ ID NO:1. In some implementations, the light chain variable region contains an amino acid sequence having about 90%, about 91%, about 92%, about 93%, about 94%, about 95%, about 96%, about 97%, about 98%, or about 99% sequence identity to SEQ ID NO:1.

In some implementations, the heavy chain variable region contains an amino acid sequence as shown in SEQ ID NO:9 or an amino acid sequence having at least 85%, at least 90%, at least 95%, or higher sequence identity to SEQ ID NO:9. In some implementations, the heavy chain variable region contains an amino acid sequence having about 90%, about 91%, about 92%, about 93%, about 94%, about 95%, about 96%, about 97%, about 98%, or about 99% sequence identity to SEQ ID NO:9.

In some implementations, the heavy chain variable region contains an amino acid sequence as shown in SEQ ID NO:33 (variable region of a humanized heavy chain version).

In some implementations, the light chain variable region contains an amino acid sequence as shown in SEQ ID NO:34 (variable region of a humanized light chain version).

In some implementations, the heavy chain variable region contains an amino acid sequence as shown in SEQ ID NO:33, and the light chain variable region contains an amino acid sequence as shown in SEQ ID NO:34.

In some implementations, the isolated monoclonal antibody or an anti-biding fragment thereof of the present disclosure is derived from a monoclonal antibody produced by a mouse hybridoma cell deposited at CGMCC under the deposit number CGMCC No. 18167 on Jun. 25, 2019. In some implementations, the isolated monoclonal antibody or an antigen-binding fragment thereof of the present disclosure binds to the same epitope on Trop2 as the monoclonal antibody produced by the mouse hybridoma cell deposited at CGMCC under the deposit number CGMCC No. 18167 on Jun. 25, 2019. In some implementations, the isolated monoclonal antibody or an antigen-binding fragment thereof of the present disclosure competes with the monoclonal antibody produced by the mouse hybridoma cell deposited at CGMCC under the deposition number CGMCC No. 18167 on Jun. 25, 2019 for binding to Trop2.

In some implementations, the isolated monoclonal antibody or an antigen-binding fragment thereof of the present disclosure specifically targets a tumor cell. The tumor cell specifically targeted by the isolated monoclonal antibody or an antigen-binding fragment thereof of the present disclosure includes but is not limited to male/female reproductive system tumor cells (such as endometrial cancer cells, uterine cancer cells, cervical cancer cells, breast cancer cells, ovarian cancer cells and prostate cancer cells), digestive system tumor cells (such as pancreatic cancer cells, colon cancer cells, gastric cancer cells, esophageal squamous cell carcinoma cells, esophageal cancer cells, cholangiocarcinoma cells and intestinal cancer cells), head and neck tumor cells (such as oral squamous cell carcinoma cells and throat cancer cells), nervous system tumor cells (such as brain glioma cells), and respiratory system tumor cells (such as lung cancer cells, for example, small cell lung cancer cells).

### III. Method for Producing Nucleic Acid, Vector and Antibody

In another aspect, the present disclosure provides an isolated nucleic acid molecule, encoding the foregoing monoclonal antibody or an antigen-binding fragment thereof of the present disclosure. In some implementations, a nucleotide sequence of the nucleic acid molecule is subjected to codon optimization for a host cell used for expression. In some implementations, the nucleic acid molecule of the present disclosure is operatively linked to an expression regulatory sequence.

In some embodiments, the nucleic acid molecule encodes CDR of a light chain variable region and a heavy chain variable region of the antibody or an antigen-binding fragment thereof of the present disclosure. Specifically, the nucleic acid molecule encodes VL CDR1, VL CDR2, VL CDR3, VH CDR1, VH CDR2, or VH CDR3. Specifically, the nucleic acid molecule has nucleotide sequences as shown in SEQ ID NO:18-20 or 26-28.

In some embodiments, the nucleic acid molecule encodes a light chain variable region or a heavy chain variable region of the antibody or an antigen-binding fragment thereof of the present disclosure. In some embodiments, the nucleic acid molecule has a nucleotide sequence as shown in SEQ ID NO:17 or a nucleotide sequence having at least 85%, at least 90%, at least 95%, or higher sequence identity to SEQ ID NO:17. In some implementations, the nucleic acid molecule has a nucleotide sequence having about 90%, about 91%, about 92%, about 93%, about 94%, about 95%, about 96%, about 97%, about 98%, or about 99% sequence identity to SEQ ID NO:17.

In some implementations, the nucleic acid molecule has a nucleotide sequence as shown in SEQ ID NO:25 or a nucleotide sequence having at least 85%, at least 90%, at least 95%, or higher sequence identity to SEQ ID NO:25. In some implementations, the nucleic acid molecule has a nucleotide sequence having about 90%, about 91%, about 92%, about 93%, about 94%, about 95%, about 96%, about 97%, about 98%, or about 99% sequence identity to SEQ ID NO:25.

In some implementations, the nucleic acid molecule has a nucleotide sequence as shown in SEQ ID NO:35.

In some implementations, the nucleic acid molecule has a nucleotide sequence as shown in SEQ ID NO:36.

The present disclosure also provides an expression vector, containing at least one foregoing nucleic acid molecule of the present disclosure.

The present disclosure also provides a host cell, transformed with at least one foregoing nucleic acid molecule or the expression vector of the present disclosure.

In another aspect, the present disclosure provides a method for producing the monoclonal antibody or an antigen-binding fragment thereof of the present disclosure, including:
(i) culturing the host cell of the present disclosure under conditions suitable for expression of the nucleic acid molecule or the expression vector, and
(ii) isolating and purifying an antibody or an antigen-binding fragment thereof expressed by the host cell.

The present disclosure also relates to an isolated antibody or an antigen-binding fragment thereof obtained by the foregoing method of the present disclosure, which is capable of specifically binding to Trop2.

In some implementations, the nucleic acid molecule of the present disclosure contains nucleotide sequences as shown in SEQ ID NO:17-20, SEQ ID NO:25-28, and SEQ ID NO:35-36.

### IV. Antibody Conjugate

The present disclosure also provides an antibody conjugate, containing the monoclonal antibody or an antigen-binding fragment thereof of the present disclosure, and a therapeutic moiety conjugated to the monoclonal antibody or an antigen-binding fragment thereof. In some embodiments, the therapeutic moiety includes a cytotoxin, a radioactive isotope, or a biologically active protein, etc.

The cytotoxin includes any agents harmful to cells (such as killer cells). For example, it includes: paclitaxel, cytochalasin B, gramicidin D, ethidium bromide, emetine, mitomycin, etoposide, teniposide, vincristine, vinblastine, colchicine, doxorubicin, daunorubicin, dihydroxyanthracinone diketone, mitoxantrone, mithramycin, actinomycin D, 1-dehydrotestosterone, glucocorticoid, procaine, tetracaine, lidocaine, propranolol, puromycin, and analogs or homologs thereof.

The therapeutic moiety that can be used for conjugation also includes, for example: antimetabolites (such as methotrexate, 6-mercaptopurine, 6-thioguanine, cytosine arabinoside, 5-fluorouracil, and decarbazine), alkylating agents (such as nitrogen mustard, chlorambucil, sarcoclorin, carmustine (BSNU) and lomustine (CCNU), cyclophosphamide, busulfan, dibromomannitol, streptozocin, mitomycin C, and cis-diamminedichloroplatinum(II) (DDP) or cisplatin), anthramycins (such as daunorubicin (formerly known as daunomycin) and doxorubicin), antibiotics (such as actinomycin D (formerly known as actinomycin), bleomycin, mithramycin, and anthramycin (AMC)), and antimitotic agents (such as vincristine and vinblastine).

Other preferred examples of therapeutic cytotoxins that can be conjugated to the antibody or fragment against Trop2 of the present disclosure include duocarmycin, calicheamicin, cantansine, auristatins, and derivatives thereof.

The linker technique in the art can be employed to conjugate the cytotoxin to the antibody of the present disclosure. Examples of linker types that have been employed to conjugate the cytotoxin to antibody against Trop2 include, but are not limited to, hydrazone, thioether, ester, disulfide, and peptide-containing linkers. For example, linkers that are susceptible to cut by low pH or by protease within lysosomal compartments can be selected. For example, a protease preferentially expressed in tumor tissues, such as cathepsin (e.g., cathepsin B, C, D), can be selected.

The antibody of the present disclosure can also be conjugated with a radioactive isotope to produce a cytotoxic radiopharmaceutical, also known as a radioactive antibody conjugate. Examples of radioactive isotopes that can be conjugated with antibodies for diagnostic or therapeutic use include, but are not limited to, iodine 131, indium 111, yttrium 90, and lutetium 177. A method for preparing radioactive antibody conjugates have been established in the art.

The antibody of the present disclosure can also be conjugated with a protein having desired biological activities, for modifying specific biological responses. Such biologically active proteins include, for example, toxins or active fragments thereof with enzymatic activity, such as abrin, ricin A, Pseudomonas exotoxin, or diphtheria toxin; proteins such as tumor necrosis factor or interferon-γ; or biological response modifiers such as lymphokines, interleukin-1 (“IL-1”), interleukin-2 (“IL-2”), interleukin-6 (“IL-6”), interleukin-10 (“IL-10”), granulocyte-macrophage colony-stimulating factor (“GM-CSF”), granulocyte colony-stimulating factor (“G-CSF”), or other immune factors such as interferon (IFN).

In some specific embodiments, the therapeutic moiety is LDM. For example, LDM contains a lipoprotein (LDP) as shown in SEQ ID NO:37, and an active enediyne (AE) chromophore that binds to LDP, as shown in Formula I. In some embodiments, LDP is linked to the antibody of the present disclosure, such as to an N-terminal of the light chain of the antibody the present disclosure, through a linker such as a linker as shown in SEQ ID NO:38.

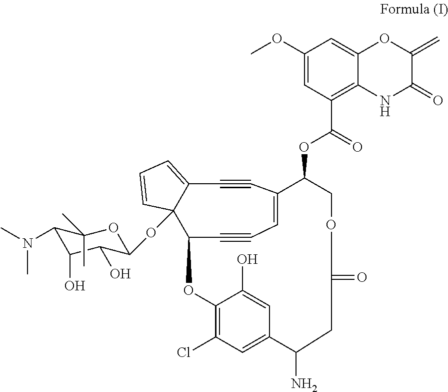 [SMILES: C=C1Oc2cc(OC)cc(C(=O)O[C@H]3COC(=O)CC(N)c4cc(O)c(c(Cl)c4)O[C@@H]4C#C/C=C/3C#CC3=CC=CC34OC3OC(C)(C)C(N(C)C)C(O)C3O)c2NC1=O]

### V. Disease Treatment and/or Prevention

The present disclosure provides a method for treating and/or preventing Trop2-related diseases such as malignant tumors in patients, including administering to the patients an effective amount of the monoclonal antibody or an antigen-binding fragment thereof against Trop2 of the present disclosure or the antibody conjugate of the present disclosure.

The Trop2-related diseases that can be treated and/or prevented by the method of the present disclosure are, for example, tumors that highly express Trop2, including but not limited to male/female reproductive system tumors, such as endometrial cancer, uterine cancer, cervical cancer, breast cancer, ovarian cancer and prostate cancer; digestive system tumors, such as pancreatic cancer, colon cancer, gastric cancer, esophageal squamous cell carcinoma, esophageal cancer, cholangiocarcinoma and intestinal cancer; head and neck tumors, such as oral squamous cell carcinoma and throat cancer; nervous system tumors, such as brain glioma; and respiratory system tumors, such as lung cancer, preferably, small cell lung cancer.

In some embodiments, the method also includes applying additional antitumor therapeutic means to the patients, such as applying a chemotherapeutic agent, an antibody targeting other tumor-specific antigens, or radiotherapy.

### VI. Pharmaceutical Composition

The present disclosure also provides a pharmaceutical composition, containing the monoclonal antibody or an antigen-binding fragment thereof against Trop2 of the present disclosure, or the antibody conjugate of the present disclosure, and a pharmaceutically acceptable vector. The pharmaceutically composition is used for treating and/or preventing Trop2-related diseases such as malignant tumors in patients.

The “pharmaceutically acceptable vector” used herein includes any and all physiologically compatible solvents, dispersing media, coatings, anti-bacterial agents and anti-fungal agents, isotonic and absorption delaying agents, etc. Preferably, the vector is suitable for intravenous, intramuscular, subcutaneous, parenteral, spinal or epidermal administration (e.g. by injection or infusion). According to administration routes, an active compound, i.e., an antibody molecule, and an immunoconjugate, can be encapsulated in a material to protect the compound from inactivating by acids and other natural conditions.

The pharmaceutical composition of the present disclosure can also contain pharmaceutically acceptable antioxidants. Examples of the pharmaceutically acceptable antioxidants include: (1) water-soluble antioxidants such as ascorbic acid, cysteine hydrochloride, sodium bisulfate, sodium metabisulfite, and sodium sulfite; (2) oil-soluble antioxidants such as ascorbyl palmitate, butylhydroxyanisole (BHA), butylated hydroxytoluene (BHT), lecithin, propyl gallate, and α-tocopherol; and (3) metal chelators such as citric acid, ethylene diamine tetraacetic acid (EDTA), sorbitol, tartaric acid, and phosphoric acid.

These compositions can also contain adjuvants such as preservatives, wetting agents, emulsifiers and dispersants.

Microorganisms can be prevented by sterilization procedures or by the inclusion of various anti-bacterial agents and anti-fungal agents such as parabens, chlorobutanol and phenol sorbates. In many cases, isotonic agents such as sugar, polyols such as mannitol, sorbitol or sodium oxide are preferably included in the composition. The delayed absorption of injectable drugs can be achieved by adding absorption delaying agents such as monostearate and gelatin into the composition.

The pharmaceutically acceptable vector includes sterile aqueous solutions or dispersion liquids, and powder agents for temporary preparation of sterile injectable solutions or dispersion liquids. The use of these media and reagents for pharmaceutically active substances is well known in the field. Conventional media or reagents, except for any that are incompatibility with the active compound, can be included in the pharmaceutical composition of the present disclosure. Supplementary active compounds can also be added into the composition.

Generally, the therapeutic composition must be sterile and stable under preparation and storage conditions. The composition can be prepared into solutions, microemulsions, liposomes, or other ordered structures suitable for high drug concentrations. The vector can be a solvent or dispersant containing, for example, water, ethanol, polyols (e.g., glycerol, propylene glycol, and liquid polyethylene glycol) and a suitable mixture thereof. For example, a coating such as lecithin can be used by maintaining the desired particle size in the case of a dispersant, and a surfactant can be used to maintain appropriate fluidity.

The sterile injectable solution can be prepared by mixing the active compound in a desired amount into a suitable solvent, and by adding one or a combination of the above ingredients as desired, followed by aseptic microfiltration. Typically, the dispersant can be prepared by mixing the active compound into a sterile vector containing the basic dispersion medium and other desired ingredients described above. The sterile powder agent for preparation of sterile injectable solutions can be preferably prepared by vacuum drying and freeze drying (lyophilization) of a sterile pre-filtered solution, so that the powder of the active ingredient plus any additional desired ingredients can be obtained.

The amount of active ingredient that can be combined with a vector material to prepare a single dosage form varies depending on the treated subject and the specific administration mode. The amount of active ingredient that can be combined with a vector material to prepare a single dose form is generally the amount of the composition that produces the therapeutic effect. Typically, on 100% basis, this amount ranges from about 0.01% to about 99% of the active ingredient, preferably from about 0.1% to about 70%, and most preferably from about 1% to about 30%, in combination with a pharmaceutically acceptable vector.

The dosing scheme can be adjusted to achieve the optimal desired response (e.g., therapeutic response). For instance, a single bolus injection can be applied, several dosages can be administered separately over time, or the dosage can be proportionally decreased or increased depending on the urgency of the therapeutic situation. It is particularly favorable to prepare parenteral compositions in the form of dosage unit that is easy to administer and uniform in dosage. The form of dosage unit used here refers to a physically discrete unit suitable for use as a unit dosage for the subject being treated; and each unit contains a predetermined amount of the active compound, which, after calculation, exerts the desired therapeutic effect in combination with the required pharmaceutical vector. The specific description for the form of dosage unit of the present disclosure is limited to and directly depends on (a) the unique characteristics of the active compound and the specific therapeutic effect to be achieved, and (b) the inherent limitations in the field for preparing such active compounds for treating individual sensitivities.

For the administration of antibody molecules, the dosage ranges from about 0.0001 to 100 mg/kg, more typically 0.01 to 20 mg/kg of the body weight of a recipient. For example, the dosage can be 0.3 mg/kg of the body weight, 1 mg/kg of the body weight, 3 mg/kg of the body weight, 5 mg/kg of the body weight, 10 mg/kg of the body weight, or 20 mg/kg of the body weight, or within the range of 1-20 mg/kg. In exemplary treatment schemes, dosing is required once a week, once every two weeks, once every three weeks, once every four weeks, once a month, once every three months, once every 3-6 months, or with a shorter initial dosing interval (such as once a week to once every three weeks) followed by a longer later dosing interval (such as once a month to once every 3-6 months).

Alternatively, antibody molecules can be administered as sustained-release preparations, in which case, a lower frequency of dosing is required. The dosage and frequency vary depending on the half-life of the antibody molecule in the patient. Generally, human antibodies exhibit the longest half-life, followed by humanized antibodies, chimeric antibodies, and non-human antibodies. The dosing amount and frequency differ depending on whether the treatment is prophylactic or therapeutic. In prophylactic applications, relatively lower dosages are administered at less frequent intervals over an extended period. Some patients receive continuous treatment for the rest of their lives. In therapeutic applications, a higher dosage can sometimes be required at shorter intervals until the progression of the disease is reduced or stopped, preferably until the disease symptoms of patient are alleviated partially or completely. After that, the patient can be dosed according to a prophylactic scheme.

The actual dosage level of the active ingredient in the pharmaceutical composition of the present disclosure can vary to obtain an amount of the active ingredient that can achieve a desired therapeutic response for a particular patient, composition, and administration mode without being toxic to the patient. The selected dosage level depends on various pharmacokinetic factors, including the activity of the applied specific composition of the present disclosure, the administration route, the administration time, the excretion rate of the applied specific compound, the duration of treatment, other drugs, compounds, and/or materials applied in combination with the applied specific composition, the age, gender, weight, condition, general health, and medical history of the patient being treated, and similar factors well known in the medical field.

The “effective amount” of the antibody or an antigen-binding fragment thereof of the present disclosure or the antibody conjugate of the present disclosure preferably results in a significant reduction in disease symptoms, an increase in the frequency and duration of asymptomatic periods of the disease, or the prevention of damage or disability caused by the disease. For example, for the treatment of tumors, compared to untreated subjects, an “effective amount” of the antibody or an antigen-binding fragment thereof of the present disclosure preferably inhibits the cell growth or tumor growth by at least about 10%, preferably at least about 20%, more preferably at least about 30%, more preferably at least about 40%, more preferably at least about 50%, more preferably at least about 60%, more preferably at least about 70%, and more preferably at least about 80%. The ability to inhibit tumor growth can be evaluated in an animal model system that can be used for predicting efficacy against human tumors, or it can be evaluated by examining the ability to inhibit cell growth, and this inhibition can be determined in vitro through tests well known to those skilled in the field. An effective amount of the antibody or an antigen-binding fragment thereof of the present disclosure can reduce tumor size, or alleviate the metastasis or recurrence of symptoms of the subject by other means, such as by preventing and/or treating. Those skilled in the field can determine such an amount based on factors such as the age of the subject, the symptom severity of the subject, and the specific composition or administration route chosen.

The antibody or an antigen-binding fragment thereof of the present disclosure, the antibody conjugate of the present disclosure, or the pharmaceutical composition of the present disclosure can be administered via one or more administration routes by utilizing one or more methods known in the field. It is to be appreciated by those skilled in the field that the administration routes and/or methods are chosen depending on the desired result. The preferred administration routes for the antibody of the present disclosure include intravenous, intramuscular, intradermal, intraperitoneal, subcutaneous, spinal, or other parenteral administration routes, such as injections or infusions. As used herein, the phrase “parenteral administration” is an administration mode other than enteral and topical administration, typically through injection, including but not limited to, intravenous, intramuscular, intra-arterial, intrasphincteric, intracapsular, intra-orbital, intracardiac, intradermal, intraperitoneal, transtracheal, subcutaneous, subepidermal, intraarticular, subcapsular, subarachnoid, intraspinal, epidural, and intra-sternal injections and infusions.

Alternatively, the antibody or an antigen-binding fragments thereof of the present disclosure, or the antibody-conjugates of the present disclosure or the pharmaceutical compositions of the present disclosure can be administered by non-parenteral routes, such as topical, epidermal, or mucosal routes, e.g., intranasal, transoral, vaginal, rectal, sublingual, or topical routes.

The active compound can be prepared with a vector that protects the compound from rapid release, such as controlled-release preparations, including implants, transdermal patches, and microencapsulated delivery systems. Biodegradable and biocompatible polymers can be used, such as ethylene vinyl acetate, polyanhydrides, polyglycolic acid, collagen, polyorthoesters and polylactic acid. Many methods for preparing such preparations are patented or generally known to those of skill in the field, seen in, e.g., Sustained and Controlled Release Drug Delivery Systems, J. R. Robinson, ed., Marcel Dekker, Inc. of New York, 1978.

In certain embodiments, the antibody of the present disclosure can be prepared to ensure proper distribution in the body. For example, the blood-brain barrier (BBB) prevents many highly hydrophilic compounds. To ensure that the therapeutic compound of the present disclosure can cross the BBB (if desired), they can be prepared, for example, in liposomes.

### VII. Combined Treatment

The antibody or an antigen-binding fragment thereof against Trop2 of the present disclosure, the antibody conjugate or the pharmaceutical composition of the present disclosure can be applied in combination with a chemotherapeutic agent or an antibody targeting other tumor antigens.

There are no particular limitations to the chemotherapeutic agent or the antibody targeting other tumor antigens that can be used in combination with the antibody of the present disclosure or the pharmaceutical composition of the present disclosure. Examples of the chemotherapeutic agent and the antibody targeting other tumor antigens include, but are not limited to: ifosfamide, cyclophosphamide, dacarbazine, temozolomide, nimustine, busulfan, melphalan, enocitabine, capecitabine, carmofur, cladribine, gemcitabine, cytosine arabinoside, tegafur, tegafur-uracil, TS-1, doxifluridine, nelarabine, hydroxyurea, fluorouracil, fludarabine, pemetrexed, pentostatin, mercaptopurine, methotrexate, irinotecan, etoposide, eribulin, sobuzoxane, docetaxel, paclitaxel, vinorelbine, vincristine, vindesine, vinblastine, actinomycin D, aclarubicin, amrubicin, idarubicin, epirubicin, zinostatin stimalamer, daunorubicin, doxorubicin, pirarubicin, bleomycin, peplomycin, mitomycin C, mitoxantrone, oxaliplatin, carboplatin, cisplatin, nedaplatin, anastrozole, exemestane, estradiol, chlormadinone, goserelin, tamoxifen, dexamethasone, bicalutamide, toremifene, flutamide, prednisolone, fosfestrol, mitotane, methyltestosterone, leuprolide, letrozole, medroxyprogesterone acetate, ibritumomab tiuxetan, imatinib, everolimus, erlotinib, gefitinib, sunitinib, cetuximab, sorafenib, dasatinib, tamibarotene, trastuzumab, retinoic acid, pembrolizumab, bevacizumab, bortezomib, and lapatinib. In one specific implementation, the chemotherapeutic agent contains platinum, such as cis-platinum.

The antibody of the present disclosure or the antibody conjugate of the present disclosure, and the chemotherapeutic agent or the antibody targeting other tumor antigens, can be administered together, or separately. In separate administration (under different administration schemes), they can be administered consecutively without interruption or administered at predetermined intervals.

There are no specific restrictions on the combined dosage of the antibody or the antibody conjugate of the present disclosure, and the chemotherapeutic agent or the antibody targeting other tumor antigens, in the pharmaceutical composition of the present disclosure. As mentioned above, the dosage of the antibody of the present disclosure can be determined by referring to the dosage when the antibody is used alone. The chemotherapeutic agent and the antibody targeting other tumor antigens can be used according to the dosage indicated for each drug or the dosage can be reduced (considering the combined effect with the antibody of the present disclosure).

The antibody of the present disclosure, the antibody conjugate of the present disclosure, or the pharmaceutical composition of the present disclosure, can also be combined with radiotherapy including applying ionizing radiation to the patients, which is applied before, during, and/or after the administration of the antibody or pharmaceutical composition of the present disclosure.

### VIII. Detection and Diagnosis

In another aspect, the present disclosure also provides a method for detecting the presence or expression level of Trop2 in a biological sample, including contacting the biological sample and a control sample with the monoclonal antibody or an antigen-binding fragment thereof against Trop2 of the present disclosure under conditions where a composition can form between the monoclonal antibody or an antigen-binding fragment thereof against Trop2 of the present disclosure and Trop2; and detecting the formation of the composition. The difference in the composition formation between the biological sample and the control sample indicates the presence or expression level of Trop2 in the sample.

In some embodiments, the biological sample is an ex vivo sample.

It has been found that Trop2 is highly expressed in many tumors, especially in epithelial malignancies. Therefore, the monoclonal antibody or an antigen-binding fragment thereof against Trop2 of the present disclosure can be used for the diagnosis of Trop2-related malignant tumors.

Therefore, the present disclosure also provides a method for detecting the presence of malignant tumors in patients, including detecting the expression level of Trop2 in a biological sample from the patient using the method of the present disclosure. The higher expression level of Trop2 in the biological sample from the patient than that in a control biological sample indicates the presence of malignant tumors in the patient.

In some embodiments, the control biological sample is derived from a healthy individual. In some embodiments, the control biological sample is derived from an individual who has been known to have no malignant tumors.

The biological sample includes, but is not limited to, a tissue sample, a blood sample, a lymph sample, etc.

In another aspect, the present disclosure provides a diagnostic agent for detecting and/or diagnosing Trop2-related diseases such as malignant tumors, containing the monoclonal antibody or an antigen-binding fragment thereof against Trop2 of the present disclosure, and an optionally physiologically acceptable vector. In some embodiments, the diagnostic agent is a contrast agent.

In another aspect, the present disclosure provides a use of the monoclonal antibody or an antigen-binding fragment thereof against Trop2 of the present disclosure in preparing a diagnostic agent for detecting and/or diagnosing Trop2-related diseases such as malignant tumors. In some embodiments, the diagnostic agent is a contrast agent.

In another aspect, the present disclosure provides a method for detecting and/or diagnosing Trop2-related diseases such as malignant tumors in a subject, including administering to the subject the monoclonal antibody or an antigen-binding fragment thereof against Trop2 of the present disclosure or the diagnostic agent of the present disclosure.

In some embodiments of the foregoing aspects of the present disclosure, the monoclonal antibody or an antigen-binding fragment of the present disclosure is also conjugated with a fluorescent dye, chemical substance, polypeptide, enzyme, isotope, and label that can be used for detection or can be detected by other reagents.

The malignant tumor includes but is not limited to male/female reproductive system tumors, such as endometrial cancer, uterine cancer, cervical cancer, breast cancer, ovarian cancer and prostate cancer; digestive system tumors, such as pancreatic cancer, colon cancer, gastric cancer, esophageal squamous cell carcinoma, esophageal cancer, cholangiocarcinoma and intestinal cancer; head and neck tumors, such as oral squamous cell carcinoma and throat cancer; nervous system tumors, such as brain glioma; and respiratory system tumors, such as lung cancer, preferably, small cell lung cancer.

### IX. Kit

A kit for the method of the present disclosure is also included in the scope of the present disclosure. The kit includes the monoclonal antibody or an antigen-binding fragment thereof of the present disclosure, the antibody conjugate of the present disclosure, the pharmaceutical composition of the present disclosure, or the diagnostic agent of the present disclosure, along with instructions for use. The kit can further include at least one additional detection reagent for detecting the presence of the monoclonal antibody of the present disclosure. The kit generally includes a label indicating the intended use and/or use method of the contents of the kit. The term “label” includes any written or recorded material provided on, with, or otherwise accompanying the kit.

### Examples

### Example 1. Establishment of Hybridoma Cell Line

### 1. Animal Immunization

Primary immunization: a Trop2 antigen was mixed with a complete Freund's adjuvant in equal volume, followed by completely ultrasonic emulsification under ice bath conditions, and intraperitoneal injection was performed on mice at 300 μL per mouse.

Second immunization: two weeks later, an antigen solution was mixed with an incomplete Freund's adjuvant in equal volume, followed by completely ultrasonic emulsification under ice bath conditions, and intraperitoneal injection was performed on mice at 300 μL per mouse.

Third immunization: the operation was the same as the second immunization, and one week later, the antibody titer in serum of the mice was measured by ELISA, with the titer in serum reaching millions.

Fourth immunization: each mouse was intraperitoneally injected with 300 μL of 50 μg antigen solution, and cell fusion was performed three days later.

### 2. Preparation of Peritoneal Macrophages

A healthy female BALB/c mouse aged 5 weeks was euthanized by cervical dislocation. The euthanized mouse was immersed and disinfected in 75% alcohol for 5 min, whose abdominal skin was dissected in a dissecting dish on a super clean worktable, exposing a peritoneal muscle layer. The peritoneal muscle was lifted with sterile forceps, and 5 mL of Roswell park memorial institute (RPMI) 1640 incomplete culture medium was injected into a peritoneal cavity. The abdomen was gently massaged for 1-2 min, a peritoneal fluid was aspirated and transferred to a 50 mL plastic centrifuge tube, and the steps were repeated 2-3 times. The peritoneal fluid was centrifuged at 1000 rpm for 5 min to remove supernatant, followed by resuspension in a 1640 complete culture medium containing hypoxanthine-aminopterin-thymidine (HAT). The resuspended fluid was placed in a 96-well culture plate and incubated at 37° C. in a 5% CO₂ incubator for one day, followed by check for contamination under a microscope. The fluid was prepared for cell fusion.

### 3. Myeloma Cell Processing

SP2/0 cells were inoculated on the back of a BALB/c mouse. When a tumor grew to about 500 mm³, it was cut and ground to prepare a cell suspension. Live cells were counted using a 0.4% trypan blue staining solution (0.1 mL of the cell suspension was added to a 0.9 mL of trypan blue staining solution for cell counting, and cells that were not stained blue were live cells). Myeloma cells with a survival rate greater than 90% can be used for fusion and kept at 37° C. for later use.

### 4. Preparation of Splenic Lymphocytes

A BALB/c mouse whose serum antibody titer had reached the fusion criterion was euthanized. The euthanized mouse was immersed and disinfected in 75% alcohol for 5 min, followed by taking the spleen on a super clean worktable and gently washing the same. The connective tissues surrounding the spleen were stripped off, and the spleen was cut into small pieces with sterile scissors. The small pieces of spleen were crushed with a plunger of a 2.5 ml syringe, followed by filtering through a 200-mesh copper net, to obtain a splenic cell suspension, which was centrifuged at 1000 rpm for 5 min. The centrifuged splenic cell suspension was washed twice with a serum-free medium, and then centrifuged as before. The suspension was taken for live cells counting using a 0.4% trypan blue staining solution, for later use.

### 5. Cell Fusion

1×10⁸ splenic cells were mixed with 2×10⁷⁻⁵×10⁷ myeloma cells SP2/0-Ag14 (usually at a ratio of 10:1 to 10:5) in a 50 mL centrifuge tube, followed by centrifugation at 1000 rpm for 5 min. Supernatant was aspirated, and the bottom of the centrifuge tube was gently tapped to loosen and evenly distribute the precipitated cells, followed by preheating at 40° C. for later use. 1 mL of a 50% PEG-1450 solution (pH 8.0) preheated to 40° C. was uniformly and slowly added over 45 s while gently rotating the centrifuge tube, allowing the PEG solution to fully and uniformly contact the loose cells. 1 mL of a serum-free medium preheated to 40° C. was aspirated and slowly and uniformly added to the bottom of the centrifuge tube over 60 s while rotating the centrifuge tube. 5 mL of the serum-free medium preheated to 40° C. was aspirated and slowly and uniformly added to the bottom of the centrifuge tube over 60 s while rotating the centrifuge tube. 10 mL of the serum-free medium preheated to 40° C. was aspirated and slowly and uniformly added to the bottom of the centrifuge tube over 90 s while rotating the centrifuge tube, which was repeated once. Centrifugation was performed at 1000 rpm for 5 min and supernatant was discarded. A 1640 complete culture medium containing HAT was added, and a cell suspension was formed by gently pipetting. The cell suspension was inoculated into 7-8 macrophage-containing 96-well plates that had no contamination after checking under a microscope, and then incubated at 37° C. in a 5% CO₂ incubator. ½ of the medium was replaced with an HAT medium after 5 days, the HAT medium was replaced with a high temperature (HT) medium after 7-10 days, and a regular complete medium can be used after 14 days. The growth of hybridoma cells was observed, and when they grew to cover more than 1/10 of the well bottom, supernatant was aspirated for antibody detection, and a second antibody detection was performed at the same interval. The results of the two positive values were compared. Wells with increased or unchanged positive values were selected, and were preliminarily determined as positive wells, and screening and cloning of the positive wells were further performed.

### 6. Screening and Subcloning of Positive Hybridoma

The dilution culture method was employed for rescreening and subcloning of hybridoma cells. Mouse peritoneal cells were prepared as above. A hybridoma cell suspension was selected from positive wells, and the hybridoma cells were diluted with an HT medium containing 20% serum to cell suspensions at different dilutions, with 2.5, 15, and 50 cells/mL. The cell suspension was placed in a 96-well plate at 0.2 mL/well, with 0.5, 3, and 10 hybridoma cells per well, respectively, and incubated at 37° C. in a 5% CO₂ incubator for 7-10 days until clones were visible. By observing under a microscope, wells with only a single clone (which was round, and had a round and smooth growth trajectory arc of edge cells) growing were selected, and were subjected to antibody detection. Cells from antibody-positive wells were taken and subjected to 2-3 rounds of dilution culture and subcloning and screening as above, the culture was expanded and the cells were stored by freezing.

### 7. Stability Verification of Hybridoma Cells

Hybridoma cell lines for detection was continuously cultured before and after cryopreservation. The antibody titer in the cell supernatant was detected by indirect ELISA. The results show no significant change in antibody titer during passages and after resuscitation, proving that the hybridoma cells can stably secrete anti-Trop2 monoclonal antibodies (FIG. 2).

This hybridoma cell has been deposited at CGMCC (No. 3, Yard 1, Beichen West Road, Chaoyang District, Beijing, Institute of Microbiology, Chinese Academy of Sciences, postal code: 100101) on Jun. 25, 2019, with the deposit number CGMCC No. 18167.

### Example 2. Preparation of Anti-Trop2 Monoclonal Antibody

### I. Antibody Preparation

Female BALB/c mice aged 7-8 weeks were selected and pre-treated with 500 μL of incomplete Freund's adjuvant via intraperitoneal injection. After 7-10 days, hybridoma cells in good growth were collected and centrifuged at 1000 rpm for 5 min, and supernatant was discarded. The hybridoma cells were resuspended in phosphate buffer saline (PBS) to prepare a cell suspension at a concentration of 1×10⁷ cells/mL. Each mouse was intraperitoneally injected with 200 μL of the suspension. After three days, the abdominal enlargement of the mice was observed daily. Ascites were typically collected 7-10 days later. The collected ascites was centrifuged at 10000 rpm for 30 min. Supernatant was collected and filtered through a 0.45 μm filter membrane to remove large lipids. The supernatant was initially purified using the ammonium sulfate precipitation method. The ascites was purified using a ProteinG (1 mL) affinity chromatography column to prepare anti-Trop2 monoclonal antibody.

### II. Antibody Identification

### 1. Antibody Purity Identification:

The purity of the anti-Trop2 monoclonal antibody was detected using HPLC. The conditions for HPLC were as follows: gel column: SEC S3000; flow rate: 1 mL/min; sample loading amount: 20 μL; mobile phase: acetonitrile gradient increased from 5% to 95% over 16 min, with a total time of 20 min; and detector: 280 nm. The purity of the prepared monoclonal antibody was determined via an integral peak area at 280 nm (FIG. 4).

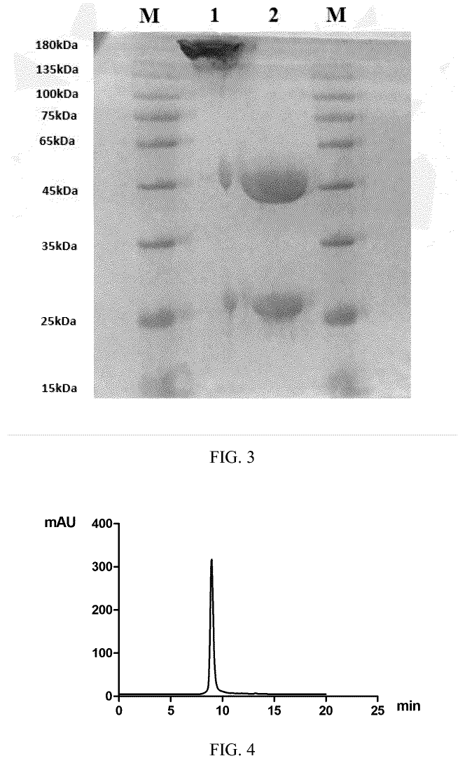

The antibody purify was detected using SDS-PAGE method: a 10 μL of anti-Trop2 monoclonal antibody sample was mixed with ¼ volume of 5× loading buffer, followed by boiling for 5 min for denaturation, for later use. A separation gel at a concentration of 12% and a spacer gel at a concentration of 5% were prepared. Electrophoresis was performed at 80 V for 30 min, and the voltage was adjusted to 120 V for 1.5 h after the sample entered the separation gel. After the electrophoresis, a gel was stained with a Coomassie blue staining solution for 3 h and then destained with a destaining solution at room temperature overnight. The gel was photographed using a gel imaging system (FIG. 3).

### 2. Antibody Class and Subclass Identification

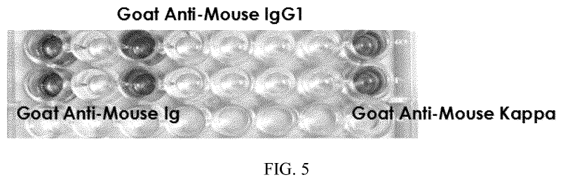

The subclass of the anti-Trop2 monoclonal antibody was identified using an SBA Clonotyping System-HRP mouse monoclonal antibody typing kit. An ELISA plate was coated with a capture antibody at a concentration of 5-10 μg/mL, with 100 μL/well, and incubated at 4° C. overnight. The plate was washed three times with phosphate buffered saline with tween (PBST), with 5 min each time. The plate was blocked with 2% bovine serum albumin (BSA) in PBST (200 μL/well) at 37° C. for 2 h and washed as before. The plate was added with an anti-Trop2 monoclonal antibody dilution solution diluted appropriately and then incubated at 37° C. for 2 h, followed by washing as above. The plate was added with various types of secondary antibodies, including Goat Anti-Mouse Ig, Human ads-HRP (positive control); Goat Anti-Mouse IgA-HRP; Goat Anti-Mouse IgG1, Human ads-HRP; Goat Anti-Mouse IgG2a, Human ads-HRP; Goat Anti-Mouse IgG2b, Human ads-HRP; Goat Anti-Mouse IgG3, Human ads-HRP; Goat Anti-Mouse IgM, Human ads-HRP; Goat Anti-Mouse Kappa-HRP; and Goat Anti-Mouse Lambda-HRP at appropriate dilutions and incubated at 37° C. for 1.5 h, followed by washing as above. The plate was added with a tetramethylbenzidine (TMB) substrate (100 μL/well) for color development in the dark at room temperature for 15 min, and the reaction was terminated with 2M H2SO2 (100 μL/well). The A450 absorbance value was measured, and positive value wells indicated the corresponding antibody subclass type (FIG. 5).

### 3. Determination of Amino Acid Sequence of Antibody

RNA was extracted from hybridoma cells, and subjected to reverse transcription to obtain a cDNA library. PCR was performed using specific primers to obtain a monoclonal antibody gene sequence, and amino acid sequences of heavy and light chains of the monoclonal antibody were obtained.

### 4. Humanization of Mouse-Derived Antibody

Design and synthesis of humanized antibody: based on a mouse-derived antibody sequence, a humanized design was performed. After the humanized antibody sequence was synthesized, a humanized antibody expression vector was constructed. Simultaneously, a chimeric human-mouse antibody expression vector was constructed as a control. Plasmid large-scale extraction was performed on the prepared expression vectors to prepare transfection-grade plasmids.

Expression and purification of humanized antibody: the prepared humanized antibody expression vector was transiently transfected into mammalian cells. The recombinant antibody was purified using Protein A, concentrated, and then quantified using the bicinchoninic acid assay (BCA) method. A target protein provided by the client was used as an antigen, coated on a 96-well plate. The binding of the humanized antibody to the target protein was detected using ELISA.

Affinity measurement of humanized antibody: the affinity of the prepared humanized antibody to the target protein was measured using BiaCoreT200.

### Example 3. Determination of Affinity Activity of Anti-Trop2 Monoclonal Antibody

### 1. Determination of Antibody-Antigen Affinity Constant by ELISA

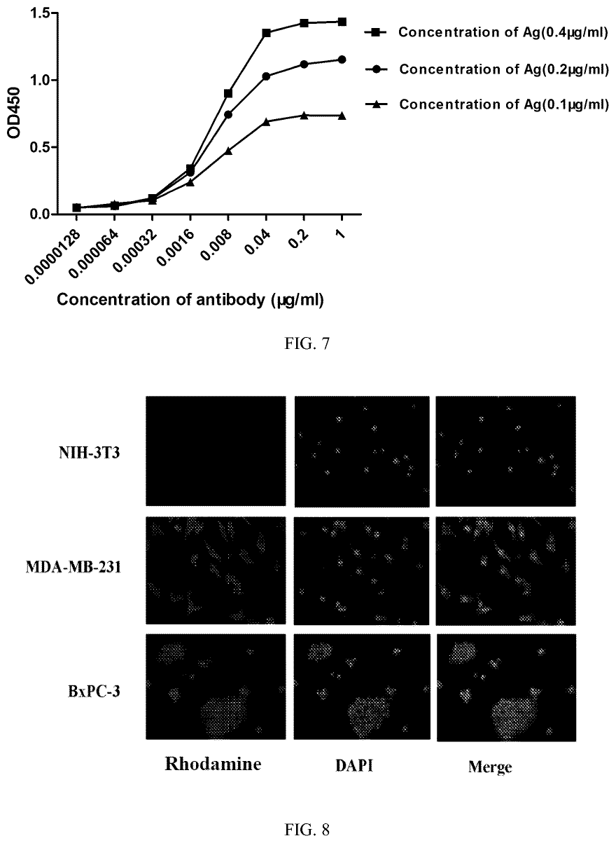

A Trop2 antigen was diluted to three dilutions (0.1 μg/mL, 0.2 μg/mL, and 0.4 μg/mL) and coated onto ELISA plates. An anti-Trop2 monoclonal antibody was diluted to concentrations of (1, 0.2, 0.04, 0.008, 0.0016, 0.00032, 0.000064, and 0.0000128 μg/mL). An antigen-antibody reaction curve was determined using an indirect ELISA. When the curve approached a plateau, it indicated that all antigens were bound, and the antibody concentration (mol/L) corresponding to 50% maximum binding to antigen was identified on the curve and substituted into the formula: Ka=(n−1)/2(n[ab]−[ab]t). The unit of Ka is M⁻¹. In this formula, [Ab] represents the antibody concentration corresponding to A=½Amax when the antigen concentration is [Ag]; [Ab]t represents the antibody concentration corresponding to A=½Amax when the antigen concentration is [Ag]t; [Ag] and [Ag]t represent the antigen concentrations; and n is the dilution factor between [Ag] and [Ag]t. (FIG. 7)

### 2. Detection of Antibody-Antigen Affinity by Biacore Method

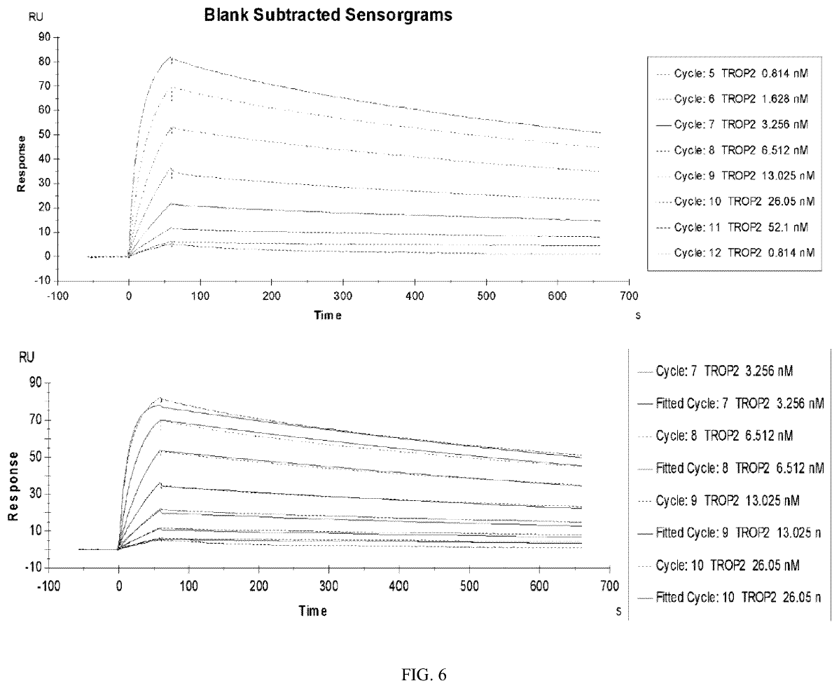

Amino of an anti-Trop2 antibody was coupled to a CM5 chip, with pH of 5.0, an antibody concentration of 10 μg/mL, sample injection time of 60 s, and flow rate of 10 μL/min, and a coupling amount of 378.6 RU. Trop2 antigen at different concentrations (0, 0.814, 1.628, 3.256, 6.512, 13.025, 26.05, and 52.1 μg/mL) was taken for flowing over the chip with a sample injection time of 60 s, flow rate of 30 μL/min, dissociation time of 600 s, and regeneration conditions of glycine-HCl, and pH 1.5 (100 s, 30 μL/min) to obtain Biacore affinity graphs and affinity constant. (FIG. 6)

### 3. Detection of Binding Ability of Antibody to Tumor Cells by Immunofluorescence

NIH3T3, MDA-MB-231, and BXPC-3 cells were cultured on cell slides at 1×10⁴ cells/well and incubated at 37° C. for 24 h. The cells after incubation were fixed with 4% paraformaldehyde for 30 min, followed by washing three times with pre-cooled PBS at 4° C., blocking with 1% BSA in PBST solution at 4° C. overnight, and adding with purified anti-Trop2 antibody at a concentration of 10 μg/mL and incubating at 4° C. overnight. An SP2/0 ascites served as a negative control. The cells were washed three times with PBST pre-cooled to 4° C., added with rhodamine-labeled goat anti-mouse IgG secondary antibody diluted 1:200 at room temperature for incubation for 1.5 h in the dark. The cells after incubation were washed three times with PBST, and one drop of 4′,6-diamidino-2-phenylindole (DAPI) (1 mg/mL) was added to each well for incubation for 10 min. Observations and photographs were taken under a fluorescence microscope. (FIG. 8)

### 4. Confocal Immunofluorescence Detection of Anti-Trop2 Antibody

HCC-827 cells in logarithmic growth phase were prepared into a single-cell suspension, after cell counting, which was inoculated into a cell slide at 1×10⁴ cells/well/200 μL, and then incubated at 37° C. for 24 h. An anti-Trop2 antibody at 10 μg/mL, 200 μL/well, was added. For the binding of antibody to the cell surface, cells were incubated after being added with antibody IMB1636 at 4° C. for 30 min. The cells were collected for washing three times with PBS, fixed with 4% paraformaldehyde (200 μL/well) for 15 min, washed three times with PBST, permeabilized with 0.2% Triton-X100 (200 μL+100 mL PBS) for 10 min, washed three times with PBST, blocked with 5% BSA at 37° C. for 30 min. The cells were added with secondary antibody (goat anti-mouse AF488) at 3 μL+1.5 mL PBST, 200 μL/well, and incubated at 37° C. for 30 min, washed five times with PBST, stained with DAPI for 15 min, washed three times with PBST, and dropwise added with an anti-fluorescence quenching agent for observation under a confocal microscope.

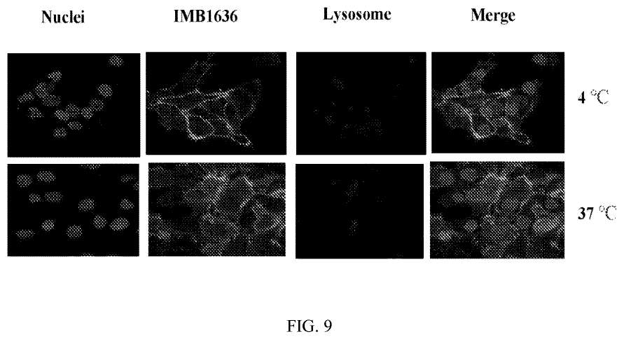

For the case of endocytosis of antibody, cells were added with antibody IMB1636 and incubated at 37° C. for 2 h. To prove lysosomal degradation of the antibody after endocytosis, LAMP-1 antibody (1:200) targeting lysosomal surface proteins was incubated at 37° C. for 2 h after cell blocking, and LAMP-1 was labeled with donkey anti-rabbit AF555 secondary antibody, making lysosomes labeled as red fluorescence. Other steps were the same as above. Co-localization of antibody IMB1636 with lysosomes was observed. (FIG. 9)

### 5. Detection of Binding Ability of Antibody to Tumor Cells by Flow Cytometry

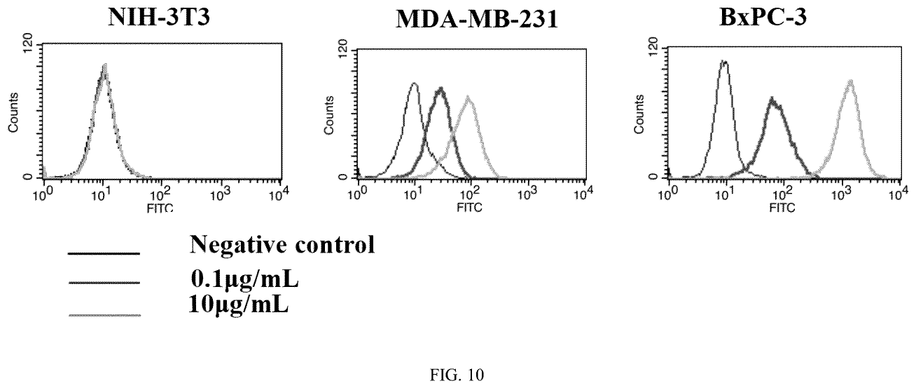

BXPC-3, MDA-MB-231, and NIH 3T3 cells were separately collected at 5×10⁶ cells/EP tube, washed with 100 μL of PBS containing 2% FBS pre-cooled to 4° C., and resuspended in 100 μL of PBS containing 2% FBS. Anti-Trop2 antibody at concentrations of 3, 1, 0.3, 0.1, 0.03, 0.01, and 0.003 μg/mL was separately added at 100 μL/tube, with SP2/0 ascite as a negative control. The cells were incubated at 4° C. for 1.5 h, washed with PBS containing 2% FBS, and added with 200 μL of fluorescein isothiocyanate (FITC)-labeled goat anti-mouse IgG secondary antibody (diluted 1:200) for incubation at 4° C. for 1 h in the dark. The cells were washed three times with PBS, and detected by flow cytometry. (FIG. 10)

### 6. Determination of Binding Ability of Antibody to Human Tumor Tissue Chip

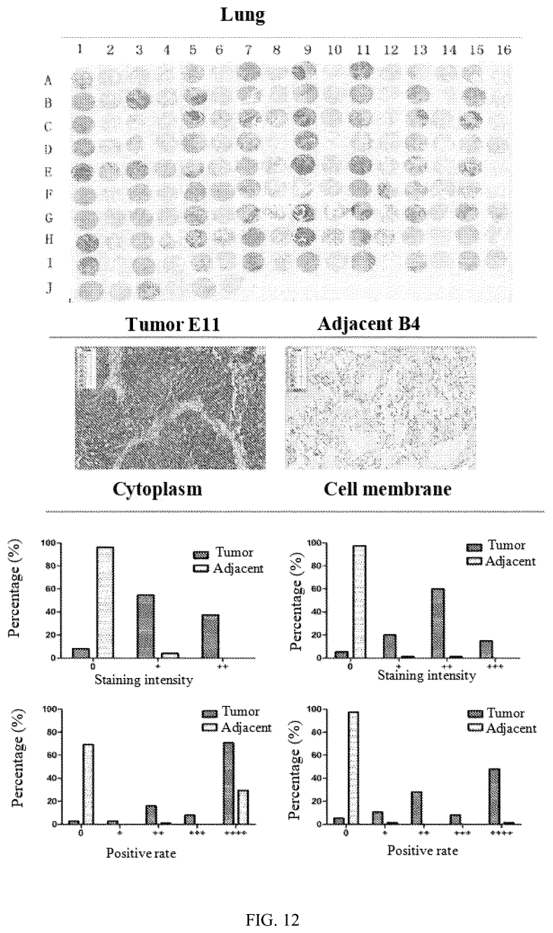

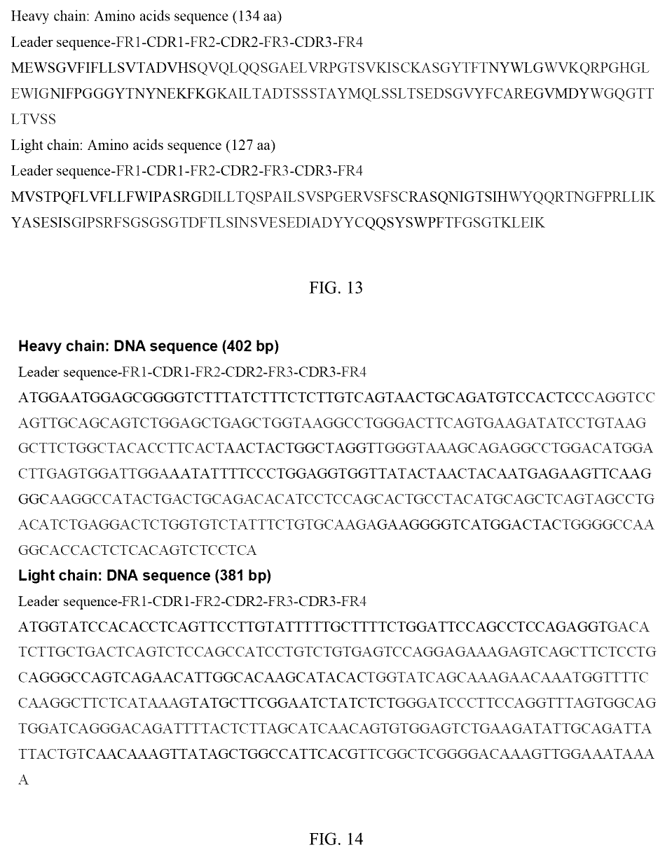

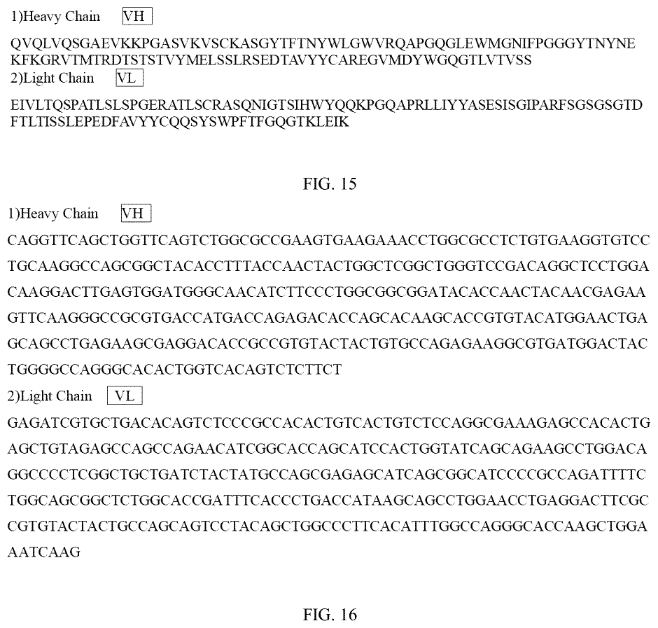

All tumor tissue chips used in this experiment were purchased from Shanghai Outdo Biotech Co., Ltd. The main experimental steps included: slide baking, dewaxing, antigen retrieval, endogenous peroxidase blocking, primary antibody incubation, secondary antibody incubation, diaminobenzidine (DAB) developing, hematoxylin counterstaining, and slide blocking. (FIG. 12)

### 7. In Vivo Imaging of Mice to Monitor the Ability of Antibody Targeting Tumors

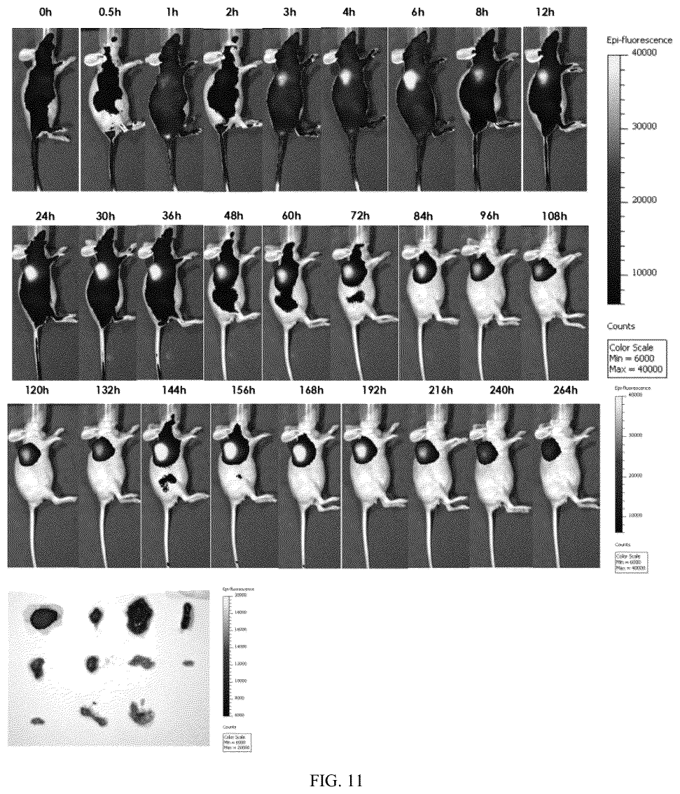

BXPC-3, MDA-MB-231, and NIH3T3 cells were inoculated in the axilla of mice at 6×10⁴ cells/mouse. When the tumor volume reached 200-300 cm², an anti-Trop2 antibody was labeled using a Dylight 680 antibody kit. The anti-Trop2 antibody was injected via tail vein into mice at 20 mg/kg. In vivo imaging was performed at 30 min, 1 h, 2 h, 3 h, 4 h, 6 h, 8 h, 10 h, 12 h, 14 h, 24 h, 30 h, 36 h, 48 h, 60 h, 72 h, 84 h, 96 h, 108 h, 120 h, 132 h, 144 h, 156 h, 168 h, 192 h, 216 h, 240 h, and 264 h. (FIG. 11)

### 8. Cytotoxic Activity of Anti-Trop2-LDM and LDM Against Tumor Cells Cultured In Vitro

Anti-Trop2-LDM was prepared, which was a conjugate of anti-Trop2 antibody with the antitumor antibiotic LDM (with the preparation method referred to: Wang R, Li L, Duan A, Li Y, Liu X, Miao Q, Gong J, Zhen Y. Crizotinib enhances anti-CD30-LDM induced antitumor efficacy in NPM-ALK positive anaplastic large cell lymphoma. Cancer Lett. 2019 Apr. 28; 448:84-93.). The cofactor protein of LDM was LDP, and an amino acid sequence of LDP was shown in SEQ ID NO:37. LDP was fused to an N-terminal of a light chain of the humanized anti-Trop2 antibody shown in SEQ ID NO: 34 via a linker shown in SEQ ID NO:38, and then formed the conjugate anti-Trop2-LDP with a heavy chain.

A LDM pore product with high activity was taken, from which, an active chromophore AE shown in Formula I was separated via a C4 column.

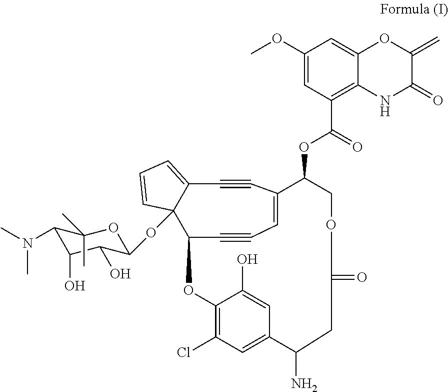 [SMILES: C=C1Oc2cc(OC)cc(C(=O)O[C@H]3COC(=O)CC(N)c4cc(O)c(c(Cl)c4)O[C@@H]4C#C/C=C/3C#CC3=CC=CC34OC3OC(C)(C)C(N(C)C)C(O)C3O)c2NC1=O]

The mobile phase was water: acetonitrile: trifluoroacetic acid at a ratio of 78%:22%:0.1%. The absorbance at 350 nm was measured, and the chromophore AE was collected. The anti-Trop2-LDP protein was mixed with the chromophore AE in a ratio of 1:4, and a mixed solution was shaken gently on a shaker at 4° C. in the dark overnight. Afterward, the mixed solution was subjected to ultrafiltration centrifugation at 4° C. and 3500 rpm for 4-6 times to remove the unassembled free chromophores. The ultrafiltration was stopped when no chromophore could be detected in the ultrafiltrated solution, and the obtained mixed solution was an antibody-drug conjugate (ADC) Anti-Trop2-LDM solution. After ultrafiltration concentration, the ADC, Anti-Trop2-LDM, was obtained. The absorbance of Anti-Trop2-LDM at 350 nm was detected using reversed-phase HPLC (C4, 300A).

Tumor cells in the logarithmic growth phase after subculture were selected and prepared into a single-cell suspension and cell counting was performed. The cell concentration was adjusted to 3×10³ cells/well, and the cells were inoculated into a 96-well plate with 80 μL/well. After 24 h of incubation, 20 μL of anti-Trop2-IgG, LDM, and Anti-Trop2-IgG-LDM diluted to different concentrations were added, with three parallel wells for each concentration. After 48 h of culture, 10 μL of CCK-8 reagent was added to each well in the dark, followed by shaking for 3 min and incubation in an incubator for 1 h. The 96-well plate was taken and shaken for 2 min, and the OD value at 450 nm was measured using a microplate reader. In addition to the drug-treated groups at different concentrations, three parallel wells each for the drug-free control group and the cell-free blank group were set up. The results are shown in the table below:

### 9. In Vivo Antitumor Effects of Anti-Trop2-LDM and LDM

IC₅₀(nM)

| Cell | Anti-Trop2-LDP | LDM |
|---|---|---|
| A431 | 0.134 ± 0.013 | 0.007 ± 0.002 |
| KYSE150 | 1.53 ± 0.188 | 0.995 ± 0.008 |
| A549 | 0.286 ± 0.047 | 0.169 ± 0.017 |
| ECA109 | 2.254 ± 0.434 | 0.445 ± 0.034 |

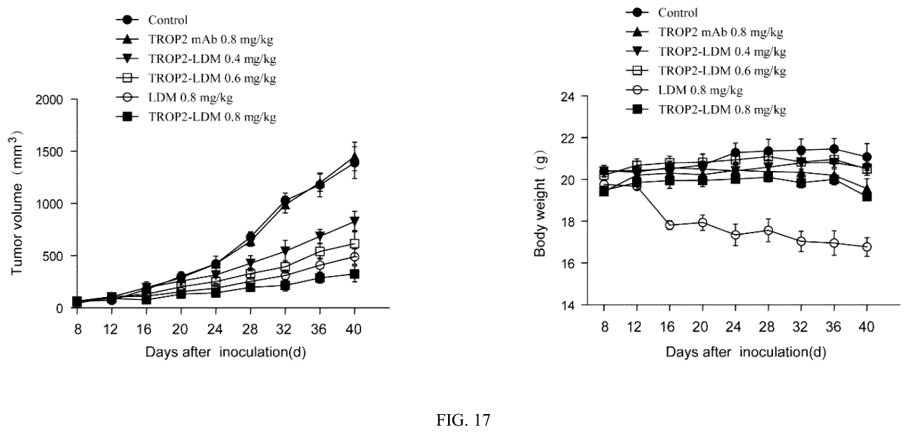

A subcutaneous transplantation tumor model was established using the human lung cancer cell line HCC827 highly expressing Trop2, to evaluate the in vivo antitumor effect of Anti-Trop2-LDM. Female nude BALB/c mice aged 6 weeks were selected and inoculated with HCC827 tumor cells at 3×10⁶ cells/mouse in the right axilla via the subcutaneous inoculation. Tumor growth was subsequently observed. When the tumor volume reached 100 mm³-150 mm³ on day 8 after inoculation, the mice were randomly divided into 6 groups, with 6 mice in each group and treated with different drugs as follows. Anti-Trop2-LDM at different concentrations (0.4 mg/kg, 0.6 mg/kg, and 0.8 mg/kg), LDM (0.05 mg/kg), and TROP2-mAb (0.8 mg/kg) were injected into the mice via tail vein once on days 8, 16, and 24, for a total of 3 doses. The control group was injected with an equal volume of normal saline. From day 8, the length (a) and width (b) of the tumor were measured every 4 days until day 40. Tumor volume was calculated using the formula V=ab²/2 to plot tumor growth curves, and the weight of the mice was measured to plot weight change curves. The results are shown in FIG. 17.

Sequence

>SEQ ID NO: 1 Trop2 light chain variable region
MVSTPQFLVFLLFWIPASRGDILLTQSPAILSVSPGERVSFSCRASQNIGTSIHWY
QQRTNGFPRLLIKYASESISGIPSRFSGSGSGTDFTLSINSVESEDIADYYCQQSYSWPFTFGS
GTKLEIK
>SEQ ID NO: 2 Trop2 VL-CDR1
RASQNIGTSIH
>SEQ ID NO: 3 Trop2 VL-CDR2
YASESIS
>SEQ ID NO: 4 Trop2 VL-CDR3
QQSYSWPFT
>SEQ ID NO: 5 Trop2 VL-FR1
DILLTQSPAILSVSPGERVSFSC
>SEQ ID NO: 6 Trop2 VL-FR2
WYQQRTNGFPRLLIK
>SEQ ID NO: 7 Trop2 VL-FR3
GIPSRFSGSGSGTDFTLSINSVESEDIADYYC
>SEQ ID NO: 8 Trop2 VL-FR4
FGSGTKLEIK
>SEQ ID NO: 9 Trop2 heavy chain variable region
MEWSGVFIFLLSVTADVHSQVQLQQSGAELVRPGTSVKISCKASGYTFTNYWL
GWVKQRPGHGLEWIGNIFPGGGYTNYNEKFKGKAILTADTSSSTAYMQLSSLTSEDSGVYF
CAREGVMDYWGQGTTLTVSS
>SEQ ID NO: 10 Trop2 VH-CDR1
NYWLG
>SEQ ID NO: 11 Trop2 VH-CDR2
NIFPGGGYTNYNEKFKG
>SEQ ID NO: 12 Trop2 VH-CDR3
EGVMDY
>SEQ ID NO: 13 Trop2 VH-FR1
QVQLQQSGAELVRPGTSVKISCKASGYTFT
>SEQ ID NO: 14 Trop2 VH-FR2
WVKQRPGHGLEWIG
>SEQ ID NO: 15 Trop2 VH-FR3
KAILTADTSSSTAYMQLSSLTSEDSGVYFCAR
>SEQ ID NO: 16 Trop2 VH-FR4
WGQGTTLTVSS
>SEQ ID NO: 17 Trop2 light chain variable region
ATGGTATCCACACCTCAGTTCCTTGTATTTTTGCTTTTCTGGATTCCAGCCTC
CAGAGGTGACATCTTGCTGACTCAGTCTCCAGCCATCCTGTCTGTGAGTCCAGGAGAAA
GAGTCAGCTTCTCCTGCAGGGCCAGTCAGAACATTGGCACAAGCATACACTGGTATCA
GCAAAGAACAAATGGTTTTCCAAGGCTTCTCATAAAGTATGCTTCGGAATCTATCTCTG
GGATCCCTTCCAGGTTTAGTGGCAGTGGATCAGGGACAGATTTTACTCTTAGCATCAAC
AGTGTGGAGTCTGAAGATATTGCAGATTATTACTGTCAACAAAGTTATAGCTGGCCATT
CACGTTCGGCTCGGGGACAAAGTTGGAAATAAAA
>SEQ ID NO: 18 Trop2 VL-CDR1
AGGGCCAGTCAGAACATTGGCACAAGCATACAC
>SEQ ID NO: 19 Trop2 VL-CDR2
TATGCTTCGGAATCTATCTCT
>SEQ ID NO: 20 Trop2 VL-CDR3
CAACAAAGTTATAGCTGGCCATTCACG
>SEQ ID NO: 21 Trop2 VL-FR1
GACATCTTGCTGACTCAGTCTCCAGCCATCCTGTCTGTGAGTCCAGGAGAAA
GAGTCAGCTTCTCCTGC
>SEQ ID NO: 22 Trop2 VL-FR2
TGGTATCAGCAAAGAACAAATGGTTTTCCAAGGCTTCTCATAAAG
>SEQ ID NO: 23 Trop2 VL-FR3
GGGATCCCTTCCAGGTTTAGTGGCAGTGGATCAGGGACAGATTTTACTCTTA
GCATCAACAGTGTGGAGTCTGAAGATATTGCAGATTATTACTGT
>SEQ ID NO: 24 Trop2 VL-FR4
TTCGGCTCGGGGACAAAGTTGGAAATAAAA
>SEQ ID NO: 25 Trop2 heavy chain variable region
ATGGAATGGAGCGGGGTCTTTATCTTTCTCTTGTCAGTAACTGCAGATGTCC
ACTCCCAGGTCCAGTTGCAGCAGTCTGGAGCTGAGCTGGTAAGGCCTGGGACTTCAGTG
AAGATATCCTGTAAGGCTTCTGGCTACACCTTCACTAACTACTGGCTAGGTTGGGTAAA
GCAGAGGCCTGGACATGGACTTGAGTGGATTGGAAATATTTTCCCTGGAGGTGGTTATA
CTAACTACAATGAGAAGTTCAAGGGCAAGGCCATACTGACTGCAGACACATCCTCCAG
CACTGCCTACATGCAGCTCAGTAGCCTGACATCTGAGGACTCTGGTGTCTATTTCTGTG
CAAGAGAAGGGGTCATGGACTACTGGGGCCAAGGCACCACTCTCACAGTCTCCTCA
>SEQ ID NO: 26 Trop2 VH-CDR1
AACTACTGGCTAGGT
>SEQ ID NO: 27 Trop2 VH-CDR2
AATATTTTCCCTGGAGGTGGTTATACTAACTACAATGAGAAGTTCAAGGGC
>SEQ ID NO: 28 Trop2 VH-CDR3
GAAGGGGTCATGGACTAC
>SEQ ID NO: 29 Trop2 VH-FR1
CAGGTCCAGTTGCAGCAGTCTGGAGCTGAGCTGGTAAGGCCTGGGACTTCA
GTGAAGATATCC TGTAAGGCTTCTGGCTACACCTTCACT
>SEQ ID NO: 30 Trop2 VH-FR2
TGGGTAAAGCAGAGGCCTGGACATGGACTTGAGTGGATTGGA
>SEQ ID NO: 31 Trop2 VH-FR3
AAGGCCATACTGACTGCAGACACATCCTCCAGCACTGCCTACATGCAGCTCA
GTAGCCTGACATCTGAGGACTCTGGTGTCTATTTCTGTGCAAGA
>SEQ ID NO: 32 Trop2 VH-FR4
TGGGGCCAAGGCACCACTCTCACAGTCTCCTCA
>SEQ ID NO: 33 Humanized heavy chain variable region
QVQLVQSGAEVKKPGASVKVSCKASGYTFTNYWLGWVRQAPGQGLEWMGNI
FPGGGYTNYNEKFKGRVTMTRDTSTSTVYMELSSLRSEDTAVYYCAREGVMDYWGQGTL
VTVSS
>SEQ ID NO: 34 Humanized light chain variable region
EIVLTQSPATLSLSPGERATLSCRASQNIGTSIHWYQQKPGQAPRLLIYYASESISG
IPARFSGSGSGTDFTLTISSLEPEDFAVYYCQQSYSWPFTFGQGTKLEIK
>SEQ ID NO: 35 Humanized heavy chain variable region
CAGGTTCAGCTGGTTCAGTCTGGCGCCGAAGTGAAGAAACCTGGCGCCTCTG
TGAAGGTGTCCTGCAAGGCCAGCGGCTACACCTTTACCAACTACTGGCTCGGCTGGGTC
CGACAGGCTCCTGGACAAGGACTTGAGTGGATGGGCAACATCTTCCCTGGCGGCGGAT
ACACCAACTACAACGAGAAGTTCAAGGGCCGCGTGACCATGACCAGAGACACCAGCAC
AAGCACCGTGTACATGGAACTGAGCAGCCTGAGAAGCGAGGACACCGCCGTGTACTAC
TGTGCCAGAGAAGGCGTGATGGACTACTGGGGCCAGGGCACACTGGTCACAGTCTCTT
CT
>SEQ ID NO: 36 Humanized light chain variable region
GAGATCGTGCTGACACAGTCTCCCGCCACACTGTCACTGTCTCCAGGCGAAA
GAGCCACACTGAGCTGTAGAGCCAGCCAGAACATCGGCACCAGCATCCACTGGTATCA
GCAGAAGCCTGGACAGGCCCCTCGGCTGCTGATCTACTATGCCAGCGAGAGCATCAGC
GGCATCCCCGCCAGATTTTCTGGCAGCGGCTCTGGCACCGATTTCACCCTGACCATAAG
CAGCCTGGAACCTGAGGACTTCGCCGTGTACTACTGCCAGCAGTCCTACAGCTGGCCCT
TCACATTTGGCCAGGGCACCAAGCTGGAAAT CAAG
>SEQ ID NO: 37 LDP amino acid sequence
APAFSVSPASGLSDGQSVSVSVSGAAAGETYYIAQCAPVGGQDACNPATATSFT
TDASGAASFSFVVRKSYTGSTPEGTPVGSVDCATAACNLGAGNSGLDLGHVALTFG
>SEQ ID NO: 38 Linker
GSGGPEGGS

1. An isolated monoclonal antibody or an antigen-binding fragment thereof against Trop2, the monoclonal antibody containing a light chain variable region and a heavy chain variable region, wherein
the light chain variable region contains:
VL CDR1, containing an amino acid sequence as shown in SEQ ID NO:2 or an amino acid sequence having 1 or 2 amino acid residue substitutions, deletions, or additions relative to SEQ ID NO: 2,
VL CDR2, containing an amino acid sequence as shown in SEQ ID NO:3 or an amino acid sequence having 1 or 2 amino acid residue substitutions, deletions, or additions relative to SEQ ID NO: 3, and
VL CDR3, containing an amino acid sequence as shown in SEQ ID NO:4 or an amino acid sequence having 1 or 2 amino acid residue substitutions, deletions, or additions relative to SEQ ID NO: 4; and
the heavy chain variable region contains:
VH CDR1, containing an amino acid sequence as shown in SEQ ID NO:10 or an amino acid sequence having 1 or 2 amino acid residue substitutions, deletions, or additions relative to SEQ ID NO:10,
VH CDR2, containing an amino acid sequence as shown in SEQ ID NO:11 or an amino acid sequence having 1 or 2 amino acid residue substitutions, deletions, or additions relative to SEQ ID NO:11, and
VH CDR3, containing an amino acid sequence as shown in SEQ ID NO:12 or an amino acid sequence having 1 or 2 amino acid residue substitutions, deletions, or additions relative to SEQ ID NO:12.

2. The isolated monoclonal antibody or an antigen-binding fragment thereof according to claim 1, wherein the monoclonal antibody contains a light chain variable region and a heavy chain variable region,
the light chain variable region containing:
VL CDR1, containing an amino acid sequence as shown in SEQ ID NO:2,
VL CDR2, containing an amino acid sequence as shown in SEQ ID NO:3, and
VL CDR3, containing an amino acid sequence as shown in SEQ ID NO:4; and
the heavy chain variable region containing:
VH CDR1, containing an amino acid sequence as shown in SEQ ID NO:10,
VH CDR2, containing an amino acid sequence as shown in SEQ ID NO:11, and
VH CDR3, containing an amino acid sequence as shown in SEQ ID NO:12.

3. The isolated monoclonal antibody or an antigen-binding fragment thereof according to claim 1, wherein the light chain variable region contains an amino acid sequence as shown in SEQ ID NO:1 or an amino acid sequence having at least 85%, at least 90%, at least 95%, or higher sequence identity to SEQ ID NO:1.

4. The isolated monoclonal antibody or an antigen-binding fragment thereof according to claim 1, wherein the heavy chain variable region contains an amino acid sequence as shown in SEQ ID NO:9 or an amino acid sequence having at least 85%, at least 90%, at least 95%, or higher sequence identity to SEQ ID NO:9.

5. The isolated monoclonal antibody or an antigen-binding fragment thereof according to claim 1, wherein the heavy chain variable region contains an amino acid sequence as shown in SEQ ID NO:33.

6. The isolated monoclonal antibody or an antigen-binding fragment thereof according to claim 1, wherein the light chain variable region contains an amino acid sequence as shown in SEQ ID NO:34.

7. The isolated monoclonal antibody or an antigen-binding fragment thereof according to claim 1, wherein the heavy chain variable region contains an amino acid sequence as shown in SEQ ID NO:33, and the light chain variable region contains the amino acid sequence as shown in SEQ ID NO:34.

8. A monoclonal antibody or an antigen-binding fragment thereof, the monoclonal antibody being produced by a mouse hybridoma cell deposited at the China General Microbiological Culture Collection Center (CGMCC), under the deposit number CGMCC No. 18167 on Jun. 25, 2019.

9. A hybridoma cell, deposited at CGMCC under the deposit number CGMCC No. 18167 on Jun. 25, 2019.

10. An antibody conjugate, containing the monoclonal antibody or an antigen-binding fragment thereof according to claim 1, and a therapeutic moiety selected from a cytotoxin, a radioactive isotope, or a biologically active protein and conjugated to the monoclonal antibody or an antigen-binding fragment thereof.

11. A pharmaceutical composition, containing the monoclonal antibody or an antigen-binding fragment thereof according to claim 1, and a pharmaceutically acceptable vector.

12. (canceled)

13. (canceled)

14. (canceled)

15. A use of the monoclonal antibody or an antigen-binding fragment thereof according to claim 1 in preparing a drug for treating and/or preventing Trop2-related diseases.

16. The use according to claim 15, wherein the Trop2-related disease is selected from male/female reproductive system tumor cells comprising endometrial cancer cells, uterine cancer cells, cervical cancer cells, breast cancer cells, ovarian cancer cells and prostate cancer cells, digestive system tumor cells comprising pancreatic cancer cells, colon cancer cells, gastric cancer cells, esophageal squamous cell carcinoma cells, esophageal cancer cells, cholangiocarcinoma cells and intestinal cancer cells, head and neck tumor cells comprising oral squamous cell carcinoma cells and throat cancer cells, nervous system tumor cells comprising brain glioma cells, and respiratory system tumor cells comprising lung cancer cells comprising small cell lung cancer cells.

17. A method for detecting the presence of malignant tumors in patients, comprising:
a) contacting a biological sample obtained from the patient with the monoclonal antibody or an antigen-binding fragment thereof according to claim 1; and
b) detecting the binding of the monoclonal antibody or an antigen-binding fragment thereof to a target antigen in the biological sample, the detected binding indicating the presence of malignant tumors in the patient.

18. The method according to claim 17, wherein the biological sample comprises a blood sample, a lymphatic sample, or a component thereof.

19. The method according to claim 17, wherein the malignant tumor is selected from male/female reproductive system tumors comprising endometrial cancer, uterine cancer, cervical cancer, breast cancer, ovarian cancer and prostate cancer, digestive system tumors comprising pancreatic cancer, colon cancer, gastric cancer, esophageal squamous cell carcinoma, esophageal cancer, cholangiocarcinoma and intestinal cancer, head and neck tumors comprising oral squamous cell carcinoma and throat cancer, nervous system tumors comprising brain glioma, and respiratory system tumors comprising lung cancer comprising small cell lung cancer.

20. An isolated nucleic acid molecule, encoding the monoclonal antibody or an antigen-binding fragment thereof according to claim 1.

21. The isolated nucleic acid molecule according to claim 20, wherein the nucleic acid molecule is operatively linked to an expression regulatory sequence.

22. The isolated nucleic acid molecule according to claim 20, wherein the nucleic acid molecule contains nucleotide sequences as shown in SEQ ID NO:17-20, SEQ ID NO:25-28, and SEQ ID NO:35-36.

23. An expression vector, containing the nucleic acid molecule according to claim 20.

24. A host cell, transformed with the nucleic acid molecule according to claim 20.

25. A method for producing a monoclonal antibody or an antigen-binding fragment thereof against Trop2, comprising:
(i) culturing the host cell according to claim 24 under conditions suitable for expression of the nucleic acid molecule or the expression vector, and
(ii) isolating and purifying an antibody or an antigen-binding fragment thereof expressed by the nucleic acid molecule or the expression vector.

26. An antibody conjugate, containing the monoclonal antibody or an antigen-binding fragment thereof according to claim 2, and a therapeutic moiety selected from a cytotoxin, a radioactive isotope, or a biologically active protein and conjugated to the monoclonal antibody or an antigen-binding fragment thereof.

27. An antibody conjugate, containing the monoclonal antibody or an antigen-binding fragment thereof according to claim 3, and a therapeutic moiety selected from a cytotoxin, a radioactive isotope, or a biologically active protein and conjugated to the monoclonal antibody or an antigen-binding fragment thereof.

28. An antibody conjugate, containing the monoclonal antibody or an antigen-binding fragment thereof according to claim 4, and a therapeutic moiety selected from a cytotoxin, a radioactive isotope, or a biologically active protein and conjugated to the monoclonal antibody or an antigen-binding fragment thereof.
